SEQUENCE LISTING (6 sequences)

SEQ ID NO: 1 - RNA, 20 nt, synthetic construct
  source 1..20: mol_type = other RNA
gtgctcctttccaggagtgg

SEQ ID NO: 2 - RNA, 20 nt, synthetic construct
  source 1..20: mol_type = other RNA
acttaaactccacgtcatcg

SEQ ID NO: 3 - DNA, 22 bp, synthetic construct
  source 1..22: mol_type = other DNA
atcggtgcgggcctcttcgcta

SEQ ID NO: 4 - RNA, 1490 nt, Homo sapiens
  source 1..1490: mol_type = mRNA
gcagataagcccagcttagcccagctgaccccagaccctctcccctcactccccccatgtcgcaggatcgagaccctgaggcagacagcccgttcaccaagccccccgccccgcccccatcaccccgtaaacttctcccagcctccgccctgccctcacccagcccgctgttccccaagcctcgctccaagcccacgccacccctgcagcagggcagccccagaggccagcacctatccccgaggctggggtcgaggctcggccccgcccctgcctctgcaacttgagcctggctgcgacccctgctctgacgtctcggaaaattcccccttgcccaggcccttgggggagggggtgcatggtatgaaatggggctgagacccccggctgggggcagaggaacccgccagagaacattcagaaggccttcatcgcatccatggacctgtggaactgggatgaggcatccccacaggaagtgcctccagggaacaagctggcagggcttgaaggagccaaattaggcttctgtttccctgatctggcactccaaggggacacgccgacagcgacagcagagacatgctggaaaggtacaagctcatccctggcaagcttcccacagctggactggggctccgcgttactgcacccagaagttccatggggggcggagcccgactctcaggctcttccgtggtccggggactggacagacatggcgtgcacagcctgggactcttggagcggcgcctcgcagaccctgggccccgcccctctcggcccgggccccatccccgccgccggctccgaaggcgccgcgggccagaactgcgtccccgtggcgggagaggccacctcgtggtcgcgcgcccaggccgccgggagcaacaccagctgggactgttctgtggggcccgacggcgatacctactggggcagtggcctgggcggggagccgcgcacggactgtaccatttcgtggggcgggcccgcgggcccggactgtaccacctcctggaacccggggctgcatgcgggtggcaccacctctttgaagcggtaccagagctcagctctcaccgtttgctccgaaccgagcccgcagtcggaccgtgccagtttggctcgatgccccaaaactaaccaccgaggtcccattcagctgtggcagttcctcctggagctgctccacgacggggcgcgtagcagctgcatccgttggactggcaacagccgcgagttccagctgtgcgaccccaaagaggtggctcggctgtggggcgagcgcaagagaaagccgggcatgaattacgagaagctgagccggggccttcgctactactatcgccgcgacatcgtgcgcaagagcggggggcgaaagtacacgtaccgcttcgggggccgcgtgcccagcctagcctatccggactgtgcgggaggcggacggggagcagagacacaataaaaattcccggtcaaacctcaaa

SEQ ID NO: 5 - DNA, 25 bp, synthetic construct
  source 1..25: mol_type = other DNA
aatcttaacaccttgtacagcccca

SEQ ID NO: 6 - DNA, 21 bp, synthetic construct
  source 1..21: mol_type = other DNA
agtgcctgctccagacttaaa

(19) United States
(12) Patent Application Publication
(10) Pub. No.: US 2023/0338420 A1
(43) Pub. Date: Oct. 26, 2023

(54) SIRPalpha Inhibited Macrophages and Neutrophils and Uses Thereof

(71) Applicant: Wisconsin Alumni Research Foundation, Madison, WI (US)

(72) Inventors: Igor Slukvin, Verona, WI (US); Aditi Majumder, Madison, WI (US); Portia Smith, Madison, WI (US); Anna Huttenlocher, Madison, WI (US); David Bennin, Mount Horeb, WI (US)

(21) Appl. No.: 18/165,244

(22) Filed: Feb. 6, 2023

Related U.S. Application Data
(60) Provisional application No. 63/306,830, filed on Feb. 4, 2022.

(51) Int. Cl. A61K 35/15; C12N 5/0786; C12N 5/0787; C12N 15/90; C12N 15/11; C12N 9/22; A61K 39/395; A61P 35/00

(52) U.S. Cl. CPC A61K 35/15; C12N 5/0645; C12N 5/0642; C12N 15/907; C12N 15/11; C12N 9/22; A61K 39/39558; A61P 35/00; C12N 2506/45; C12N 2500/90; C12N 2501/115; C12N 2501/15; C12N 2501/155; C12N 2501/165; C12N 2501/22; C12N 2501/145; C12N 2501/2303; C12N 2501/2306; C12N 2310/20; C12N 2800/80

(57) ABSTRACT

The disclosure generally relates to methods for producing macrophages and neutrophils serum-free and feeder-free conditions from SIRPα inhibited pluripotent stem cells. The disclosure further relates to SIRPα inhibited macrophages and neutrophils and uses thereof.

### RELATED APPLICATION

This application claims priority to U.S. provisional application No. 63/306,830, filed Feb. 4, 2022, the disclosure of which is expressly incorporated by reference herein.

### STATEMENT REGARDING FEDERALLY SPONSORED RESEARCH

This invention was made with government support under HL142665 and OD011106 awarded by the National Institute of Health. The government has certain rights in the invention.

### INCORPORATION BY REFERENCE OF SEQUENCE LISTING PROVIDED ELECTRONICALLY

This application contains a Sequence Listing submitted as an electronic text file named “21-1677-US_sequence_listing.xml” having a size in bytes of 7.62 kb, and created on Feb. 6, 2023. The information contained in this electronic file is hereby incorporated by reference in its entirety.

### FIELD OF THE DISCLOSURE

The disclosure generally relates to methods for producing macrophages and neutrophils in serum-free and feeder-free conditions from SIRPα inhibited pluripotent stem cells. The disclosure further relates to SIRPα inhibited macrophages and neutrophils and uses thereof.

### BACKGROUND

Current cancer immunotherapies concentrate on targeting cancer-specific antigens by the use of chimeric antigen receptors (CAR) T cells or monoclonal antibodies (mAb). There is an urgent need to develop novel immunotherapies that can treat patients with solid tumor cancers. Indiscriminately, solid cancers contain extremely complex tumor microenvironments (TME) that circumvent the innate and adaptive immune systems' anti-tumoral mechanisms. The heterogeneous nature of the tumor and the ability to shield from the immune system are the main reasons why current immunotherapies have not had much success targeting solid cancers. CAR-T cells and mAbs rely on the specificity of the cancer antigen which limits the targeting abilities to mainly circulatory cancers. Other mechanisms such as T cell exclusion, hypoxic milieu, and immunosuppressive myeloid cells create a hostile environment for the adaptive immune system to have any robust anti-tumorigenic effect (Joyce & Fearon (2015) Science (New York, N.Y), 348(6230), 74-80).

Macrophages are a group of diverse and active immune cells that are found across the body as either tissue-resident macrophages (TR-Macs) or circulating monocyte-derived macrophages (MD-Macs) (Davies, et al., (2013) Nature Immunology, 14(10), 986-995). Developing from the common myeloid progenitor lineage, macrophages are innate antigen presenting immune cells with an incredible appetite for phagocytosis. Upon activation, macrophages polarize into pro-inflammatory M1 subtype, or anti-inflammatory M2 subtype in which there are many other subtypes on a polarization spectrum (Kielbassa, et al., (2019) Frontiers in Immunology, 10:2215). Within the TME, tumor cells recruit macrophages via chemokine signaling, such as the CCL2/CCR2 pathway, and polarize the macrophages into a pathological, pro-tumorigenic state called tumor associated macrophages (TAMs) (Chen, et al., (2019) Journal of Biomedical Science, 26(1), 78). Through analyzing the role of macrophages in the TME, it has been found that TAMs mainly resemble M2 macrophages with increased anti-inflammatory and wound-healing signatures (Chanmee, et al., (2014) Cancers, 6(3), 1670-1690). TAMs produce many inhibitory factors including TGF-beta, IL-10, Arg1, IDO, and HIF-1alpha that inhibit and starve the existing T cell population within the TME (Zhu, et al., (2021) Journal of Cancer, 12(1), 54-64). The tumor cells within the TME circumvent the anti-tumorigenic properties of macrophages by the upregulation of immunosuppressive signals, including “don't eat me” cell receptors. One of the “don't eat me” receptors, CD47, was discovered by Weissman and colleagues as a mechanism for both hematological and solid tumor cancer cells to escape phagocytosis by TAMs (Chao, et al., (2010) Cell, 142(5), 699-713; Majeti, et al., (2009) Cell, 138(2), 286-299; Willingham, et al., (2012) Proceedings of the National Academy of Sciences, 109(17), 6662-6667). This cell surface receptor on tumor cells is recognized by the myeloid-specific receptor: signal regulatory protein alpha (SIRPα). SIRPα is abundantly expressed in macrophages, dendritic cells, and neutrophils. SIRPα is a ligand for the ubiquitously expressed “don't-eat-me” signal molecule CD47. SIRPα also promotes M2 polarization of tumor-associated macrophages. In physiological state, CD47 is expressed ubiquitously on normal, healthy cells as a marker of self; notably red blood cells (RBC) express CD47 to prevent macrophages from phagocytosing them as they mature (Oldenborg, et al., (2000) Science (New York, NY), 288(5473), 2051-2054; Oldenborg, et al., (2001) The Journal of Experimental Medicine, 193(7), 855-862). When CD47 and SIRPα interaction occurs, a signal is transduced to the immunoreceptor tyrosine-based inhibitory motifs (ITIMs) on the cytosolic tail of SIRPα. The ITIMs recruit SHP-1 and SHP-2 domain-containing phosphatases which block Myosin IIa and subsequent cytoskeletal rearrangement (Murata, et al., (2018) Cancer Science, 109(8), 2349-2357). Therefore, CD47/SIRPα activation specifically blocks phagocytosis from occurring when activating phagocytic stimuli is present.

Due to the vast upregulation of CD47 across many solid tumor cancers, targeting this signaling pathway by knocking out SIRPα in macrophages to generate cellular immunotherapy has the potential for widespread cancer treatment. In addition, macrophages have unique advantages for use cellular immunotherapy due to their access to the TME, phagocytic capabilities, and direct communication to the adaptive immune system via antigen presentation. Deriving macrophages from human donor peripheral blood monocytic cells (PBMCs) would be tedious and inefficient for genetic alteration and clinical use. Accordingly, there is a need in the art for efficient and cost-effective protocols.

### SUMMARY

Provided herein is a method of producing modified macrophages from pluripotent stem cells, the method comprising:
(a) culturing human pluripotent stem cells having inhibited expression of signal regulatory protein alpha (SIRPα) in normoxic conditions for about 24 hours in serum-free culture medium comprising of L-ascorbic acid-2-phosphate magnesium, sodium selenium, transferrin, insulin, NaHCO₃, fibroblast growth factor 2 (FGF2), transforming growth factor beta 1 (TGFβ1), and a Rho kinase (ROCK) inhibitor;
(b) further culturing the human pluripotent stem cells of (a) in hypoxia conditions for about 48 hours in serum-free culture medium comprising bone morphogenetic protein 4 (BMP4), FGF2, Activin A, an inhibitor of glycogen synthase 3 (GSK3), and a ROCK inhibitor to induce mesoderm formation;
(c) further culturing the cultured cells of (b) in hypoxic conditions for about 48 hours in serum-free culture medium comprising FGF2, a vascular endothelium growth factor (VEGF), and an inhibitor of TGFβ-mediated signaling to induce hemogenic endothelium formation;
(d) further culturing the cultured cells of (c) in normoxic conditions for about 6 days in serum-free culture medium comprising FGF2, a VEGF, stem cell factor (SCF), thrombopoietin (TPO), interleukin-6 (IL-6), and interleukin-3 (IL-3), wherein the hemogenic endothelium differentiate into HPCs;
(e) culturing the HPCs of (d) in normoxic conditions for about 6 days in serum-free culture medium comprising macrophage colony-stimulating factor (M-CSF), IL-3, and IL-6 to obtain myeloid progenitors and monocytic cells; and
(f) further culturing the cultured cells of (e) in normoxic conditions for about 4 days in serum-free culture medium comprising M-CSF, whereby the cultured myeloid progenitors and monocytes differentiate into a cell population comprising modified macrophages.

In particular embodiments the inhibitor of TGFβ-mediated signaling is SB431542. In other particular embodiments the inhibitor of GSK3 is lithium chloride (LiCl). In certain embodiments the ROCK inhibitor used in the practice of the methods disclosed herein is Y-27632.

In certain embodiments the pluripotent stem cells are induced pluripotent stem cells (iPSCs).

Advantageously expression of SIRPα is inhibited in the human pluripotent stem cells by gene mutation, RNA-mediated inhibition, RNA editing, DNA gene editing or base editing. Particular gene editing methods useful in the practice of these methods use a nuclease that includes but are not limited to a meganuclease, zinc-finger nucleases (ZFNs), transcription activator-like effector nucleases (TALENs), and Cas enzyme, particularly embodiments using Cas9. Advantageously gene editing results in knocking out SIRPα expression.

Also provided herein are modified macrophages produced according to the disclosed methods

Further provided herein is a method of producing modified macrophages from pluripotent stem cells, the method comprising:
(a) transiently introducing exogenous ETV2 in human pluripotent stem cells having inhibited expression of SIRPα and culturing the ETV2-induced pluripotent stem cells in serum-free culture medium comprising FGF-2 to produce a population of ETV2-induced hematoendothelial progenitor cells (ETV2-induced HEPs);
(b) culturing the ETV2-induced HEPs in serum-free and xeno-free culture medium comprising granulocyte-macrophage colony-stimulating factor (GM-CSF) and FGF2 for a sufficient time to produce non-adherent myeloid progenitors;
(c) culturing the non-adherent myeloid progenitors in serum-free and xeno-free culture medium comprising M-CSF, IL-6, and IL-3; and
(d) further culturing the cultured cells of (c) in serum-free and xeno-free culture medium comprising M-CSF for a sufficient time to differentiate the non-adherent myeloid progenitors into modified macrophages.

In particular embodiments, step (a) of the method comprises culturing the ETV2-induced pluripotent stem cells for about 1-2 days; step (b) of the method comprises culturing the ETV2-induced HEPs for about 6-7 days; and step (c) and step (d) comprises culturing myeloid progenitor cells for about 9 to 10 days.

In particular embodiments the serum-free and xeno-free culture medium in step (b) further comprises UM171.

In certain embodiments the pluripotent stem cells are induced pluripotent stem cells (iPSCs).

Advantageously expression of SIRPα is inhibited in the human pluripotent stem cells by gene mutation, RNA-mediated inhibition, RNA editing, DNA gene editing or base editing. Particular gene editing methods useful in the practice of these methods use a nuclease that includes but are not limited to a meganuclease, zinc-finger nucleases (ZFNs), transcription activator-like effector nucleases (TALENs), and Cas enzyme, particularly embodiments using Cas9. Advantageously gene editing results in knocking out SIRPα expression.

Also provided herein are modified macrophages produced according to these disclosed methods.

Further provided herein is a method of producing modified neutrophils from pluripotent stem cells, the method comprising:
(a) transiently introducing exogenous ETV2 in human pluripotent stem cells having inhibited expression of SIRPα and culturing the ETV2-induced pluripotent stem cells in serum-free culture medium comprising FGF-2 to produce a population of ETV2-induced hematoendothelial progenitor cells (ETV2-induced HEPs);
(b) culturing the ETV2-induced HEPs in serum-free and xeno-free culture medium comprising GM-CSF and FGF2 for a sufficient time to produce non-adherent myeloid progenitors; and
(c) culturing the non-adherent myeloid progenitors in serum-free and xeno-free culture medium comprising granulocyte-colony stimulating factor (G-CSF) and retinoic acid receptor agonist for a time sufficient to differentiate the myeloid progenitors into neutrophils.

In particular embodiments the serum-free and xeno-free culture medium in step (b) further comprises UM171. In particular embodiments the retinoic acid receptor agonist is AM580.

In particular embodiments, step (a) of the method comprises culturing the ETV2-induced pluripotent stem cells for about 1-2 days; step (b) of the method comprises culturing the ETV2-induced HEPs for about 6-7 days; and step (c) and step (d) comprises culturing myeloid progenitor cells for about 8 to 9 days.

Advantageously expression of SIRPα is inhibited in the human pluripotent stem cells by gene mutation, RNA-mediated inhibition, RNA editing, DNA gene editing or base editing. Particular gene editing methods useful in the practice of these methods use a nuclease that includes but are not limited to a meganuclease, zinc-finger nucleases (ZFNs), transcription activator-like effector nucleases (TALENs), and Cas enzyme, particularly embodiments using Cas9. Advantageously gene editing results in knocking out SIRPα expression.

Also provided herein are modified neutrophils produced according to these disclosed methods.

Also provided herein are methods for treating cancer by administering a therapeutically effective amount of the modified macrophages produced by the methods disclosed herein. In certain embodiments these methods also include administering a tumor-specific antibody. Also provided are methods for treating cancer by administering a therapeutically effective amount of the modified neutrophils produced by the methods disclosed herein.

In certain embodiments the neutrophils produced by these methods are useful for treating an infection, in particular a bacterial infection and particularly advantageously wherein the bacterial infection is a systematic infection.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

FIG. 1A-1D show the generation of SIRPα-Knockout (KO) induced pluripotent stem cells (iPSCs). FIG. 1A is a schematic of CRISPR/Cas9 driven knockout of SIRPα gene at exon 3 using two sgRNAs. FIG. 1B shows DNA extractions for genomic PCR on an agar gel for each clone. Specifically, nucleofection of hiPSCs with sgRNAs and Cas9 protein was performed. After several days, clones were selected and expanded. FIG. 1C shows phase-contrast images of WT and SIRPα-KO iPSCs. FIG. 1D shows a western blotting of collected cell lysates from WT and SIRPα-KO iPSCs differentiated into macrophages.

FIG. 2A-2E show hematopoietic differentiation of SIRPα-KO iPSCs. FIG. 2A shows a 2D monolayer differentiation schematic for generation of iPSC-Macrophages. FIG. 2B shows phase-contrast microscope images taken on day 6, 7, and 9 of differentiation to show the floating hematopoietic progenitors (HPs) arise from the hemogenic endothelium. FIG. 2C shows the number of HPs generated from a singular iPSC, which was calculated by: (# of live floating cells generated on day 9/# of iPSCs plated on day −1). A students t-test was performed to calculate the p-value at alpha=0.05. FIGS. 2D & 2E show day 9 floating cells collected and stained for flow cytometry analysis. Gates were drawn from FMO controls and compensation was performed in FlowJo software using single color controls with BD beads.

FIG. 3A-3F show SIRPα-KO iPSC-myeloid cells. FIG. 3A shows cytospin and Wright-Giemsa stain of day 15 WT and SIRPα-KO iPSC-myeloid cells. Confocal microscope images were taken. FIGS. 3B and 3C show Day 15 WT and SIRPα-KO iPSC-myeloid cells collected for flow cytometry analysis for CD45, CD14, CD11b, CD16, and CD18. The cells were gated on single, live cells using Ghost Dye 540 and FlowJo software. The populations were plotted and a two-way ANOVA was performed in Prism 9, showing no significance between the WT and SIRPα-KO (n=5). FIG. 3D shows M1 and M2 polarization schematic. FIG. 3E shows phase contrast images of M0, M1, and M2 i-Macs taken for morphology. FIG. 3F shows unstained (top), M0 (second), M1 (third), and M2 (bottom) i-Macs collected and analyzed in flow cytometry for CD14, CD263, CD206, CD86, CD80, and HLA-DR. Flow cytometry dot plots and histograms were made in FlowJo 10.

FIG. 4A-4G show that SIRPα-KO i-Macs have superior phagocytosis of CD47+ cancer cells. FIG. 4A shows WT and SIRPα-KO i-Macs co-cultured with SKOV3 GFP+ cancer cells in 2:1, 4:1, 8:1 effector to target ratios, SKOV3 cell number remaining the same. The cells were either cultured with or without anti-HER2 monoclonal antibody, then collected for flow cytometry analysis 24 hours later. Percentage (%) of phagocytosed SKOV3 cells was calculated by: (# of GFP+ CD45+ cells/# of total GFP+ cells)×100. Flow plots are representative of two independent experiments. FIG. 4B shows a chart depicting mean and SEM of % phagocytosed SKOV3 cells after 24-hour co-culture created in Prism 9. FIG. 4C shows a fluorescent microscopy image that was performed 1 hour post co-culture to capture active phagocytosis, shown by the white arrows. White cells are SKOV3-GFP+and grey cells are unstained SIRPα-KO i-Macs. FIG. 4D shows a chart depicting mean and SEM of % phagocytosed WM266-4 cells after 6-hour co-culture created in Prism 9. FIG. 4E shows a fluorescent microscopy image that was performed 1 hour post co-culture to capture active phagocytosis, shown by the white arrows. White cells are WM266-4 stained with Cell Trace Violet and grey cells are unstained SIRPα-KO i-Macs. FIGS. 4F and 4G show iMacs co-cultured with GFP-expressing SKOV-3 ovarian cancer for 6 hours and analyzed by flow cytometry using CD45 APC antibody. FIG. 4F depicts the percentage (%) of GFP+ cells depleted by iMacs through phagocytosis. FIG. 4G depicts the percentage (%) of actively phagocytosing iMacs out of the total pool of iMacs. For statistical analyses, two-way ANOVA tests were used to determine significance with alpha=0.05.

FIG. 5A-5J show that SIRPα-KO i-Macs are cytotoxic against MCF7 cancer spheroids with anti-HER2 monoclonal antibody. FIG. 5A shows MCF7 spheroids co-cultured with 5×10⁶ i-Macs/mL for 4 days. C/PI staining was performed post co-culture to assess live and dead cells. Dead cells are highlighted with white arrows. FIG. 5B shows the number of dead cells per spheroid counted by hand under microscope and graphed. FIG. 5C-FIG. 5E show the results when iMacs were co-cultured with GFP-Luc2+SKOV-3 or WM266-4 cells for 24, 48, or 96 hours and analyzed by SpectraMax plate reader to evaluate bioluminescence. FIG. 5C depicts antibody-dependent cellular cytotoxicity (ADCC) of SKOV-3 cells at 48 hours. FIG. 5D depicts analysis at only 10:1 effector to target ratio. FIG. 5E depicts ADCC of WM266-4 cells at 48 hours. For statistical analyses, two-way ANOVA tests were used to determine significance with alpha=0.05. FIG. 5F-FIG. 5J show results illustrating SIRPα-KO and wildtype (WT) iMacs that were either cultured alone or co-cultured with SKOV-3 ovarian cancer for 48 hrs and supernatants were collected for Human Inflammation 20-Plex ProcartaPlex Panel. Supernatants were tested in technical duplicates and raw values were produced on MAGPIX xMAP instrument plotted against a standardized curve. FIG. 5F shows a heatmap that depicts normalized expression of 20 different cytokines across all groups. (shown in FIG. 5G-5J). Plots represent means of concentration (pg/mL) of IL-1beta, IL-1alpha, IFN-gamma and TNF-alpha within supernatant samples.

FIG. 6A-6C show ETV2 mmRNA hematopoietic differentiation and macrophage generation. FIG. 6A shows the ETV2 mmRNA differentiation schematic from day 0-19. FIG. 6B shows the flow cytometry analysis of CD45, CD206 CD163, CD80, CD11b and CD18 expression in macrophages generated from ETV2 mmRNA transfected IISH2i-BM9 hiPSCs on day 19. FIG. 6C is a representative image of Wright stained cytospins showing the morphology of macrophages.

FIG. 7 shows CD47 expression on WM266-4. WM266-4 cancer cells were collected and analyzed by flow cytometry with anti-human CD47-PE antibody. WM266-4 cells are right histogram and unstained control is left histogram.

FIG. 8 is a schematic diagram of the ETV2 mmRNA constructs disclosed herein.

FIG. 9 is a schematic diagram of the methods disclosed herein for generation of wild type and SIRPα^{-/-} neutrophils in defined serum free and feeder free conditions using ETV2 mmRNA.

FIG. 10 shows representative phase contrast images showing the difference in morphology during the hematoendothelial development and neutrophil differentiation following transduction wild type and SIRPα^{-/-} hiPSCs with ETV2 mmRNA.

FIG. 11 shows the flow cytometric analysis of CD45 expression in myeloid progenitors generated from ETV2 mmRNA transfected wild type and SIRPA^{-/-} hiPSCs on day 9.

FIG. 12A-12H show the flow cytometric analysis of CD45, CD16 and CD11b expression in generated wild type and SIRPα^{-/-} neutrophils. FIG. 12G-12H show representative images of Wright staining showing the morphology of wild type and SIRPα^{-/-} neutrophils.

FIG. 13 shows the results of an in vitro cytotoxicity assay of neutrophils generated from wild type and SIRPα^{-/-} hiPSCs.

FIG. 14A shows the analysis of phagocytosis of pHrodo Green E. coli particles by wild type (WT) vs SIRPA^{-/-} (SIRPα) neutrophils. The bar graph is from 3 independent experiments showing fold change of cells from 37° C. incubation with phagocytosed acidified E. coli bio-particles. Bars show±SEM. Differences in lines are statistically significant (p=0.0385) as determined by unpaired t test. FIG. 14B shows the analysis of the Candida auris planktonic yeast kill assay. The percentage of viable yeast was quantified relative to yeast with no neutrophils. Neutrophil-only controls were subtracted from that value. Bar graph show±SEM from 3 independent experiments showing percent yeast survival. Differences compared to wild type were not significant (p=0.1755) as determined by unpaired t test. FIG. 14C shows the 123 DHR ROS production from iPSC neutrophils of cells treated with 50 ng/ml PMA. Graph bars show±SEM from three independent experiments. Fold change was calculated from initial time to two hours incubation. Difference of wild type vs SIRPA^{-/-} (SIRPα) were not significant (p=0.2102) as determined by unpaired t test. FIG. 14D shows the quantification of chemotactic index and velocity of iPSC neutrophils, showing significant differences of iPSC neutrophils exposed to the chemoattractant fMLP. Graph bars show±SEM from three independent experiments. P values for chemotactic index 0.0312 and velocity in um/min<0.0001 as determined by unpaired t test.

### DETAILED DESCRIPTION

The disclosure generally relates to methods for producing macrophages and neutrophils in serum-free and feeder-free conditions from SIRPα inhibited pluripotent stem cells. The disclosure further relates to SIRPα inhibited macrophages and neutrophils and uses thereof.

All publications, including but not limited to patents and patent applications, cited in this specification are herein incorporated by reference as though set forth in their entirety in the present application.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

“Pluripotent stem cells” refer to cells having the capacity to differentiate into cells of all three germ layers. “Embryonic stem cells” or “ESCs” mean a pluripotent cell or population of pluripotent cells derived from an inner cell mass of a blastocyst. See Thomson et al., (1998) Science 282:1145-1147.

“Induced pluripotent stem cells” or “iPS cells” refer to a pluripotent cell or population of pluripotent cells that may vary with respect to their differentiated somatic cell of origin, that may vary with respect to a specific set of potency-determining factors and that may vary with respect to culture conditions used to isolate them, but nonetheless are substantially genetically identical to their respective differentiated somatic cell of origin and display characteristics similar to higher potency cells, such as ESCs. See, e.g., Yu et al., (2007) Science 318:1917-1920.

“Hematopoietic precursor cells (HPCs)” and “hematopoietic progenitors (HPs)” refer to immature multipotent progenitor cells of the hematopoietic lineage. HPCs are characterized by surface expression of CD45 and, in some cases, CD34, and a capacity to differentiate into myeloid and lymphoid progenitors and terminally differentiated lymphoid and myeloid cells.

“Myeloid progenitors” are cells capable of differentiating into cell types of the myeloid lineages.

“Chemically-defined culture” “fully defined, growth factor free culture conditions,” and “fully-defined conditions” indicate that the identity and quantity of each medium ingredient is known and the identity and quantity of supportive surface is known.

“Xeno-free culture medium” refers to medium that does not contain any components derived from animal sources, such as, for example, serum.

As used herein, an “SIRPα knockout” or “SIRPα-KO” is intended to encompass any disruption or deletion of SIRPA gene that results in the dysfunction or nonfunction of the SIRPα protein as it pertains to binding to CD47 or other SIRPα binding partners and/or as it pertains to the signaling within the SIRPα-expressing cell. This definition includes the insertion of an external plasmid/gene within the SIRPA gene locus or deleting a portion or the entire region of the SIRPA gene.

### Methods for Production of Macrophages Using Morphogen-Driven Differentiation Systems

The methods provided herein comprise differentiating human pluripotent stem cells under conditions that promote differentiation of the pluripotent stem cells into hematopoietic progenitor cells and macrophages.

In some embodiments provided herein is a method of producing modified macrophages from pluripotent stem cells, the method comprising:
(a) culturing human pluripotent stem cells having inhibited expression of signal regulatory protein alpha (SIRPα) in normoxic conditions for about 24 hours in a serum-free culture medium comprising of L-ascorbic acid-2-phosphate magnesium, sodium selenium, transferrin, insulin, NaHCO₃, fibroblast growth factor 2 (FGF2), transforming growth factor beta 1 (TGFβ1), and a Rho kinase (ROCK) inhibitor;
(b) further culturing the human pluripotent stem cells of (a) in hypoxia conditions for about 48 hours in a serum-free culture medium comprising bone morphogenetic protein 4 (BMP4), FGF2, Activin A, an inhibitor of glycogen synthase 3 (GSK3), and a ROCK inhibitor to induce mesoderm formation;
(c) further culturing the cultured cells of (b) in hypoxic conditions for about 48 hours in a serum-free culture medium comprising FGF2, a vascular endothelium growth factor (VEGF), and an inhibitor of TGFβ-mediated signaling to induce hemogenic endothelium formation;
(d) further culturing the cultured cells of (c) in normoxic conditions for about 6 days in a serum-free culture medium comprising FGF2, a VEGF, stem cell factor (SCF), thrombopoietin (TPO), interleukin-6 (IL-6), and interleukin-3 (IL-3), wherein the hemogenic endothelium generates HPCs;
(e) culturing the HPCs of (d) in normoxic conditions for about 6 days in a serum-free culture medium comprising macrophage colony-stimulating factor (M-CSF), IL-3, and IL-6 to generate myeloid progenitors and monocytes; and
(f) further culturing the cultured cells of (e) in normoxic conditions for about 4 days in a serum-free culture medium comprising M-CSF, whereby the cultured myeloid progenitors and monocytes differentiate into a cell population comprising modified macrophages.

Suitable pluripotent cells for use herein include human embryonic stem cells (hESCs) and human induced pluripotent stem (iPS) cells. ESCs are commercially available from sources such as WiCell Research Institute (Madison, Wis.). In particular embodiments, the pluripotent stem cells are induced pluripotent stem cells.

The pluripotent stem cells used in the methods disclosed herein have inhibited expression of SIRPα. SIRPα is abundantly expressed in macrophages, dendritic cells, and neutrophils. SIRPα is a ligand for the ubiquitously expressed “don't-eat-me” signal molecule CD47. SIRPα also promotes M2 polarization of tumor-associated macrophages.

“Having inhibited expression of SIRPα,” indicates the gene is repressed or not expressed in a functional protein form. In particular embodiments, the expression of SIRPα is knocked out such that there is no expression of SIRPα. This inhibition or knockout can be obtained by gene mutation, RNA-mediated inhibition, RNA editing, DNA gene editing or base editing.

In particular embodiments, the gene editing method comprises the use of a nuclease selected from a meganuclease, zinc-finger nucleases (ZFNs), transcription activator-like effector nucleases (TALENs), and Cas enzyme. In particular embodiments, the nuclease is a Cas9 enzyme.

In some embodiments, normoxic conditions refer to conditions where oxygen is provided at or about standard atmospheric levels. In some embodiments, normoxic conditions refer to oxygen conditions of about 15% to about 20% oxygen (e.g., about 15%, 16%, 17%, 18%, 19%, 20% O₂).

In some embodiments, hypoxic conditions refer to a level of environmental oxygen (e.g., a cell culture incubator gas mixture) of about 3% Otto about 10% O₂. In some embodiments, hypoxic conditions are about 5% O₂.

In some embodiments, the culture is “serum-free” which refers to cell culture materials that are free of serum obtained from animal or human (e.g., fetal bovine) blood.

In some embodiments, the culture conditions are feeder-free, meaning that the culture does not use feeder cells. In particular embodiments, the culture conditions are serum-free and feeder-free.

In particular embodiments, the method disclosed herein comprises an attachment step comprising culturing human pluripotent stem cells in a culture medium in normoxic conditions (i.e., where oxygen is provided at or about standard atmospheric levels) for about 24 hours. In particular embodiments, the culture medium is E8-TeSR. “E8 culture medium” and “E8” are used interchangeably and refer to the chemically defined culture medium having the following defined components: DMEM/F12, L-ascorbic acid-2-phosphate magnesium, sodium selenium, and NaHCO₃, transferrin, insulin, FGF2 and TGFβ1.

In some embodiments, the culture medium used for culturing hPSCs to HPCs and/or myeloid progenitors to macrophages is “IF9S” media, which comprises IMDM/F12, L-ascorbic acid 2-phosphate Mg²⁺ salt, monothioglycerol, sodium selenite, polyvinyl alcohol, Glutamax™, non-essential amino acids (NEAA), chemically defined lipid concentrate (Life Technologies; Cat. No. 1905031), Holo-Transferrin, and insulin.

In particular embodiments, the culture medium comprises a Rho kinase (ROCK) inhibitor. Rock inhibitors, which are known in the art and include, but are not limited to, for example, Y27632 (commercially available from Stem Cell Technologies), and those found in Liao J K, Seto M, Noma K. Rho kinase (ROCK) inhibitors. J Cardiovasc Pharmacol. 2007; 50(1):17-24, the contents of which are incorporated by reference in its entirety.

In some embodiments, the inhibitor of TGFβ-mediated signaling is SB431542. In some embodiments, the inhibitor of GSK3 is lithium chloride (LiCl).

Also provided herein is a population of modified macrophages produced by the methods disclosed herein. The modified macrophages have inhibited expression of SIRPα as well as superior anti-tumor activity for therapeutic purposes.

### Uses of Macrophages and Neutrophils Using ETV2 Modified mRNA Differentiation Systems

The present disclosure also provides methods for efficient macrophage and neutrophil production from pluripotent stem cells using direct programming with transient expression of ETV2, e.g., by addition of modified mRNA (mmRNA) of ETV2 into the hiPSCs. Initially, hiPSCs are directly programmed into hematoendothelial progenitors using ETV2 mmRNA which transiently produced ETV2 within the cells. Next, the hematoendothelial progenitors are then differentiated into myeloid progenitors in the presence of GM-CSF, FGF2 and optionally UM171 (the presence of UM171 in combination with GM-CSF and FGF2 increases the number of macrophages neutrophils produced by the methods). Myeloid progenitors which are non-adherent could be continuously collected from cultures every 8-10 days for up to 30 days of post ETV2 transfection. Finally, these myeloid progenitors are subsequently differentiated into macrophages or neutrophils. The methods for macrophage and neutrophil production from pluripotent stem cells using direct programming with transient expression of ETV 2 are described in U.S. Publication No. 20200385676, the contents of which are incorporated by reference in its entirety.

In particular embodiments disclosed herein is a method of producing modified macrophages from pluripotent stem cells, the method comprising:
(a) transiently introducing exogenous ETV2 in human pluripotent stem cells having inhibited expression of SIRPα and culturing the ETV2-induced pluripotent stem cells in serum-free and xeno-free culture medium comprising FGF-2 to produce a population of ETV2-induced hematoendothelial progenitor cells (ETV2-induced HEPs);
(b) culturing the ETV2-induced HEPs in serum-free and xeno-free culture medium comprising granulocyte-macrophage colony-stimulating factor (GM-CSF) and FGF2 for a sufficient time to produce non-adherent myeloid progenitors;
(c) culturing the non-adherent myeloid progenitors in serum-free and xeno-free culture medium comprising M-CSF, IL-6, and IL-3; and
(d) further culturing the cultured cells of (c) in serum-free and xeno-free culture medium comprising M-CSF for a sufficient time to differentiate the non-adherent myeloid progenitors into modified macrophages.

In particular embodiments provided herein is a method of producing modified neutrophils from pluripotent stem cells, the method comprising:
(a) transiently introducing exogenous ETV2 in human pluripotent stem cells having inhibited expression of SIRPα and culturing the ETV2-induced pluripotent stem cells in serum-free and xeno-free culture medium comprising FGF-2 to produce a population of ETV2-induced hematoendothelial progenitor cells (ETV2-induced HEPs);
(b) culturing the ETV2-induced HEPs in serum-free and xeno-free culture medium comprising GM-CSF and FGF2 for a sufficient time to produce non-adherent myeloid progenitors; and
(c) culturing the non-adherent myeloid progenitors in serum-free and xeno-free culture medium comprising granulocyte-colony stimulating factor (G-CSF) and retinoic acid receptor agonist for a time sufficient to differentiate the myeloid progenitors into neutrophils.

Suitable pluripotent cells for use herein include human embryonic stem cells (hESCs) and human induced pluripotent stem (iPS) cells. ESCs are commercially available from sources such as WiCell Research Institute (Madison, Wis.). In particular embodiments, the pluripotent stem cells are induced pluripotent stem cells.

The pluripotent stem cells used in the methods disclosed herein have inhibited expression of SIRPα.

“Having inhibited expression of SIRPα,” indicates the gene is repressed or not expressed in a functional protein form. In particular embodiments, the expression of SIRPα is knocked out such that there is no expression of SIRPα. This inhibition or knockout can be obtained by gene mutation, RNA-mediated inhibition, RNA editing, DNA gene editing or base editing.

In particular embodiments, the gene editing method comprises the use of a nuclease selected from a meganuclease, zinc-finger nucleases (ZFNs), transcription activator-like effector nucleases (TALENs), and Cas enzyme. In particular embodiments, the nuclease is a Cas9 enzyme.

ETV2 can be transiently introduced into the PSCs by methods known in the art. Methods of transiently expressing ETV2 in PSCs are known in the art, and include, but are not limited to, for example, introducing transiently exogenous nucleic acids encoding the protein of interest (e.g., by plasmid expression vector transfection, or modified mRNA transfection); protein transduction, among others. In one embodiment, mmRNA of ETV-2 (e.g., Accession No: NM_014209.2; SEQ ID NO:4) is introduced into the PSCs by suitable methods. Methods of transiently expressing ETV2 in PSCs are described in U.S. Pat. No. 9,382,531, the contents of which are incorporated by reference in its entirety. Methods of introducing mmRNA into PSCs are known in the art, and include, but are not limited to, the method described in the Examples, for example, by transfection or electroporation. The methods of introducing mmRNA or DNA to transiently express ETV-2 protein is within the skill of one in the art and are not limited to what is demonstrated in the examples

After initiating transient expression of ETV2 in the hPSCs, these cells are cultured for a sufficient time to produce a population of ETV2-induced hematoendothelial progenitor cells (ETV2-induced HEPs). In particular embodiments, a sufficient time is a period of about 24 hours to about 4 days. In some embodiments, a sufficient amount of time to produce a population of ETV2-induced hematoendothelial progenitor cells comprises culturing the ETV2-induced cells for about 1-2 days. In some embodiments, a sufficient amount of time to produce a population of ETV2-induced hematoendothelial progenitor cells comprises culturing the ETV2-induced cells for about 3-8 days, for example, for about 4 days. For example, in some embodiments, the step to produce a population of ETV2-induced hematoendothelial progenitor cells comprises culturing for 3 days, alternatively 4 days, alternatively 5 days, alternatively 6 days, alternatively 7 days, alternatively 8 days to produce ETV2-induced hematoendothelial progenitor cells.

In some embodiments, the culture medium is “serum-free” which refers to cell culture materials that are free of serum obtained from animal or human (e.g., fetal bovine) blood.

In some embodiments, the culture conditions are feeder-free, meaning that the conditions do not use feeder cells. In particular embodiments, the culture conditions are serum-free and feeder-free.

In particular embodiments, the methods disclosed herein use a maintenance culture medium for culturing the hPSCs after transfection with ETV2 mmRNA. In particular embodiments the culture medium is E8-TeSR. E8 culture medium” and “E8” are used interchangeably and refer to the chemically defined culture medium having the following defined components: DMEM/F12, L-ascorbic acid-2-phosphate magnesium, sodium selenium, and NaHCO₃ in a final volume of 200, transferrin, insulin, FGF2 and TGFβ1.

In some embodiments, the culture medium is a xeno-free cell culture medium. In some embodiments, the culture condition is xeno-free, serum free and feeder-free.

Serum-free and xeno-free medium suitable for culturing the ETV2-induced hematoendothelial progenitor cells to produce non-adherent myeloid progenitors are known in the art and include, but are not limited to, for example, StemLine II (commercially available from Sigma Aldrich).

In some embodiments, a sufficient amount of time for culturing the ETV2-induced HEPs to produce non-adherent myeloid progenitors is at least 4 days, for example, at least 4-23 days. In some embodiments, a sufficient amount of time for culturing the ETV2-induced HEPs to produce non-adherent myeloid progenitors is about 6 to 7 days.

In some embodiments, the methods disclosed herein comprise isolating the non-adherent myeloid cells from the culture. Suitable methods of isolating the cells are known in the art. In one example, non-adherent cells can be collected from the culture leaving the adherent cells behind. In some embodiments, the adherent cells isolated from the non-adherent myeloid cells, may be used in methods of producing macrophages.

For production of macrophages, the methods comprise the step of culturing the myeloid progenitors in a culture medium comprising M-CSF, IL-6, and IL-3 and further culturing the cultured cells comprising M-CSF for a sufficient time to differentiate the myeloid progenitors into modified macrophages. A suitable time to differentiate the myeloid progenitors into modified macrophages includes for at least 9 days, for example at least 9-21 days. In some embodiments, a suitable time for culturing the myeloid progenitors to differentiate into modified macrophages is about 9 to 10 days.

Serum-free and xeno-free medium suitable for culturing the myeloid progenitors to differentiate into modified macrophages are known in the art and include, but are not limited to, for example, StemLine II (commercially available from Sigma Aldrich).

For production of neutrophils, the methods comprise the step of culturing the myeloid progenitors in a culture medium comprising granulocyte-colony stimulating factor (G-CSF) and retinoic acid receptor agonist for a sufficient time to differentiate the myeloid progenitors into modified neutrophils. In some embodiments the retinoic acid receptor agonist is AM580.

A suitable time to differentiate the myeloid progenitors into modified neutrophils includes for at least 9 days, for example at least 9-21 days. In some embodiments, a suitable time for culturing the myeloid progenitors to differentiate into modified macrophages is about 9 to 10 days.

Serum-free and xeno-free medium suitable for culturing the myeloid progenitors to differentiate into modified neutrophils are known in the art and include, but are not limited to, for example, StemSpan™ H3000 (StemCell Technologies).

Also provided herein is a population of modified macrophages produced by the methods disclosed herein. The modified macrophages have inhibited expression of SIRPα as well as superior anti-tumor activity for therapeutic purposes.

Also provided herein is a population of modified neutrophils produced by the methods disclosed herein. The modified neutrophils have inhibited expression of SIRPα as well as superior anti-bacterial and anti-tumor activity for therapeutic purposes. While the methods disclosed herein include ordered, sequential events, the timing of the events may be varied by at least 20%. For example, while a particular step may be disclosed in one embodiment as lasting one day, the event may last for more or less than one day. For example, “one day” may include a period of about 18 to about 30 hours. Periods of time indicated that are multiple day periods may be multiples of “one day,” such as, for example, two days may span a period of about 36 to about 60 hours, and the like. In another embodiment, time variation may be lessened, for example, where day 2 is 48+/−3 hours from d0; day 4 is 96+/−3 hours from d0, and day 5 is 120 hours+/−3 hours from d0.

### Methods for Use of the Modified Macrophages and Neutrophils

In particular embodiments, SIRPα knockout macrophages and neutrophils disclosed herein are useful for treating or preventing various disorders such as a cancer or an infection. In particular embodiments provided herein is a method of treating cancer comprising administering the SIRPα knockout macrophages disclosed herein. In particular embodiments provided herein is a method of treating cancer comprising administering the SIRPα knockout neutrophils disclosed herein. In some embodiments, the method comprises administering the SIRPα knockout macrophages disclosed herein together with a tumor-specific antibody.

In particular embodiments provided herein is a method of treating an infection comprising administering the SIRPα knockout neutrophils disclosed herein. In some embodiments, the infection is a systematic infection.

The terms “treatment” or “treat,” as used herein, refer to both therapeutic treatment and prophylactic or preventative measures. Those in need of treatment include subjects having cancer as well as those prone to having cancer or those in cancer is to be prevented. In some embodiments, the methods, compositions, and combinations disclosed herein can be used for the treatment of cancer. In other embodiments, those in need of treatment include subjects having an infection as well as those prone to have an infection or those in which an infection is to be prevented. In certain embodiments, the methods, compositions, and combinations disclosed herein can be used for the treatment of infections.

In some cases, macrophages and neutrophils obtained according to a method provided herein can be administered as a pharmaceutical composition comprising a therapeutically effective amount of macrophages and neutrophils as a therapeutic agent (i.e., for therapeutic applications).

The terms “pharmaceutical composition” or “therapeutic composition,” as used herein, refer to a compound or composition capable of inducing a desired therapeutic effect when properly administered to a subject. In some embodiments, the disclosure provides a pharmaceutical composition comprising a pharmaceutically acceptable carrier and a therapeutically effective amount of the macrophages or neutrophils of the disclosure.

The terms “pharmaceutically acceptable carrier” or “physiologically acceptable carrier,” as used herein, refer to one or more formulation materials suitable for accomplishing or enhancing the delivery of the macrophages or neutrophils of the disclosure.

The term “subject” is intended to include human and non-human animals, particularly mammals. In certain embodiments, the subject is a human patient.

The terms “administration” or “administering,” as used herein, refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Generation of SIRPA Knockout Macrophages and Characterization Thereof

### Materials and Method

### hiPSC Maintenance

hiPSCs were cultured on Matrigel-treated 6-well plates with mTeSR media in normoxic conditions. Media was changed every day until cells grew to 60-70% confluency. Cells were dissociated into small clumps by treatment with EDTA and passaged 1:6 into a new Matrigel-treated 6-well plate.

### Generating SIRPα-Knockout iPSCs

hiPSCs were grown to 60-70% confluency on Matrigel-coated plates in mTeSR1 media and isolated in a single cell suspension by treatment with TrypLE. Cas9 protein and sgRNAs (gRNA1: GUGCUCCUUUCCAGGAGUGG (SEQ ID NO: 1) and gRNA2: ACUUAAACUCCACGUCAUCG (SEQ ID NO: 2)) were diluted in Stem cell Nucleofector Solution 2 and incubated on ice for 20 mins. 10⁵ cells were diluted into the Cas9/sgRNA solution and immediately placed in a cuvette for electroporation using Lonza Nucleofector 2b device on setting A-23. The cells were serially diluted into 6-well Matrigel-coated plate with mTeSR media supplemented with 1× CloneR (STEMCELL Technologies). After 10-14 days, colonies formed from single cells were isolated and expanded in separate wells of a 24-well plate. After the clones were 60-70% confluent, cells were collected by EDTA-PBS dissociation for cryopreservation and isolating DNA extracts for genomic PCR. Each clone was genotyped by using SIRPA primers: αAATCTTAACACCTTGTACAGCCCCA (SEQ ID NO: 5) and AGTGCCTGCTCCAGACTTAAA (SEQ ID NO: 6).

### hiPSC Hematopoietic Differentiation

The day prior to differentiation, single cell suspension of hiPSCs were acquired by TrypLE treatment of 60-80% confluent iPSCs. Washed single cells were plated on Collagen IV-treated 6-well plates at a density of 3000 cells/cm² in TeSR-E8 media with 10 uM Rho kinase inhibitor (Y-27632 Tocris) and placed in the normoxic incubator. On day 0 and thereafter, component-defined IF9S media was used. To initiate differentiation into early mesoderm, hiPSCs were treated with BMP4 (50 ng/ml), FGF2 (50 ng/ml), Activin A (15 ng/ml), LiCl (2 mM), and 10 uM Rho kinase inhibitor (Y-27632 Tocris) on Day 0 and placed in hypoxic conditions (5% oxygen) for 48 hours. To continue differentiation into the hematovascular mesoderm, a full media change was performed on Day 2 and cells were treated with FGF2 (50 ng/ml), VEGF (50 ng/ml), and SB431542 (5 uM) and placed in hypoxic conditions for an additional 48 hours. On days 4 and 6, cells were treated with FGF2 (50 ng/ml), VEGF (50 ng/ml), SCF (50 ng/ml), TPO (50 ng/ml), IL3 (long/ml), and IL6 (50 ng/ml) to continue differentiation into hemogenic endothelial and hematopoietic progenitors (HPs) until day 9 in normoxic conditions. On day 9, floating hematopoietic progenitors (HPs)/hematopoietic precursor cells (HPCs) were collected for flow cytometry analysis or further differentiation.

### hiPSC Macrophage Generation

For macrophage generation, IF9S media and normoxic conditions were used throughout. To initiate myelopoiesis, day 9 floating hematopoietic progenitors were plated on ultra-low attachment (ULA) 6-well plates (Corning Costar) at a density of 1×10⁵ live HPs/well and treated with M-CSF (80 ng/ml), IL-3 (long/ml) and IL-6 (50 ng/ml) for 72 hours. On day 12, cells were treated with additional media containing M-CSF, IL-3 and IL-6 for another 72 hours to generate monocytic cell clumps. On day 15, terminal macrophage differentiation was performed by collecting all cells from the ULA 6-well plates and resuspending in fresh media treated with 80 ng/ml M-CSF and plated at a density of 2×10⁵ cells/well on uncoated tissue culture 6-well plates for 96 hours. On day 19, macrophages were collected by vigorous pipetting to dissociate any adhered cells to be used for analysis and experiments.

### mmRNA Synthesis and Transfection

Human ETV2 transcript variant 1 (NM_014209.3) was cloned into a 5′-MCS-1β construct as described previously (Suknuntha et al., 2018, Stem Cell Rev. 14, 525-534). To generate IVT templates with a 180-A tract, a reverse primer containing 180 T base pairs and an ATCGGTGCGGGCCTCTTCGCTA (SEQ ID NO: 3) forward primer including T7 promoter were used in a PCR reaction. All PCR reactions were carried out using Phusion (Thermo Fisher Scientific). The mmRNA was synthesized using the MEGAscript T7 Kit (Ambion, Austin, TX), using a custom ribonucleoside cocktail comprised of 3′-0-Me-m7G(5′)ppp(5′)G ARCA cap analog, pseudouridine triphosphate (TriLink BioTechnologies, San Diego, CA), ATP, guanosine triphosphate, and cytidine triphosphate. The synthesis reactions were set up according to the manufacturer's instructions. Reactions were incubated for 2 h at 37° C. and treated with DNAse. RNA was purified using a PureLink RNA Mini Kit (Thermo Fisher Scientific) and adjusted with RNase-free water to 100 ng/μL working concentration before being stored at −80° C. Undifferentiated hiPSCs were transfected with using TransIT-mRNA reagent in E8 medium containing ROCK inhibitor (Suknuntha et al., 2018, Stem Cell Rev. 14, 525-534). In brief, for transfection, single-cell suspension was prepared using HyQtase (Thermo Fisher Scientific). Per one well of transfection, a total of 2×10⁵ cells in 1 mL complete E8 medium with 10 μM ROCK inhibitor (STEMCELL Technologies) were plated into a collagen IV-coated 6-well plate; 30-60 min later, a mixture of 200 ng ETV2: TranslT-mRNA (Minis Bio, Madison, WI) was added to each well according to the manufacturer's instructions.

### ETV2-mmRNA Generation of Macrophages from hiPSCs

The day after transfection (day 1), the medium was changed with 1 mL of Stemline II (Sigma) supplemented with 20 ng/mL of human FGF2 (PeproTech). On day 2, 1 mL of the same medium was added. On day 3, the medium was changed and 1 mL of Stemline II supplemented with FGF2 (20 ng/mL), GM-CSF (25 ng/mL) (PeproTech), and UM171 (50 nM; Xcess Biosciences) were added. This medium was added daily up to days 8. On day 9, floating cells were gently harvested and used for macrophage differentiation. To induce specification to monocyte/macrophage lineages, floating cells were cultured in Stemline II, supplemented with M-CSF (80 ng/mL; Amgen), IL6 (50 ng/mL; Amgen), IL3 (10 ng/mL; Amgen) at 1×10⁵ cells/well density in ultra-low attachment 6 well plate. After 3 days, 2 mL of the same medium with all components and cytokines was added on the top of existing culture. At day 15, to induce macrophage differentiation, cells were transferred to a 6 well plate containing Stemline II, supplemented with M-CSF (80 ng/mL; Amgen) for 4 days. At day 19, macrophages were gently harvested and filtered through a 70-μM mesh (Falcon, Life Sciences) before analysis.

### Results

### Generation of SIRPα-Knockout iPS Cell Line

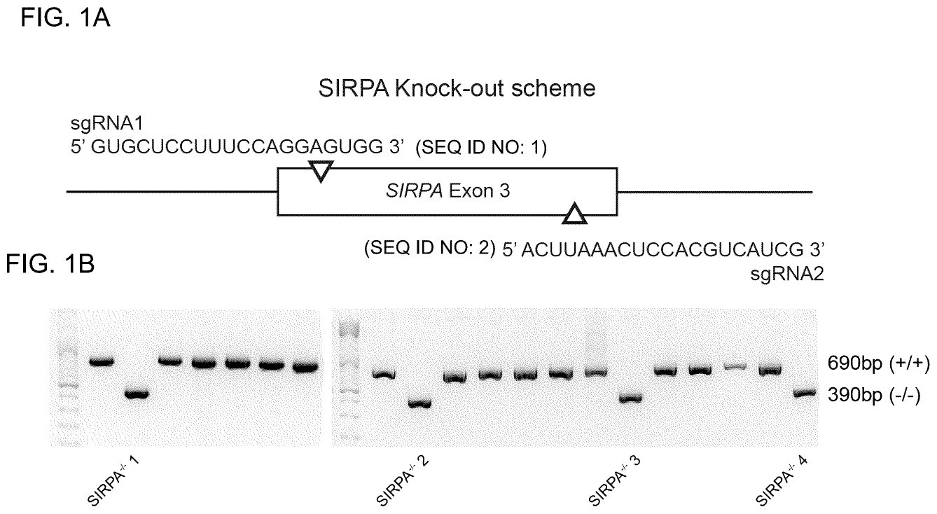

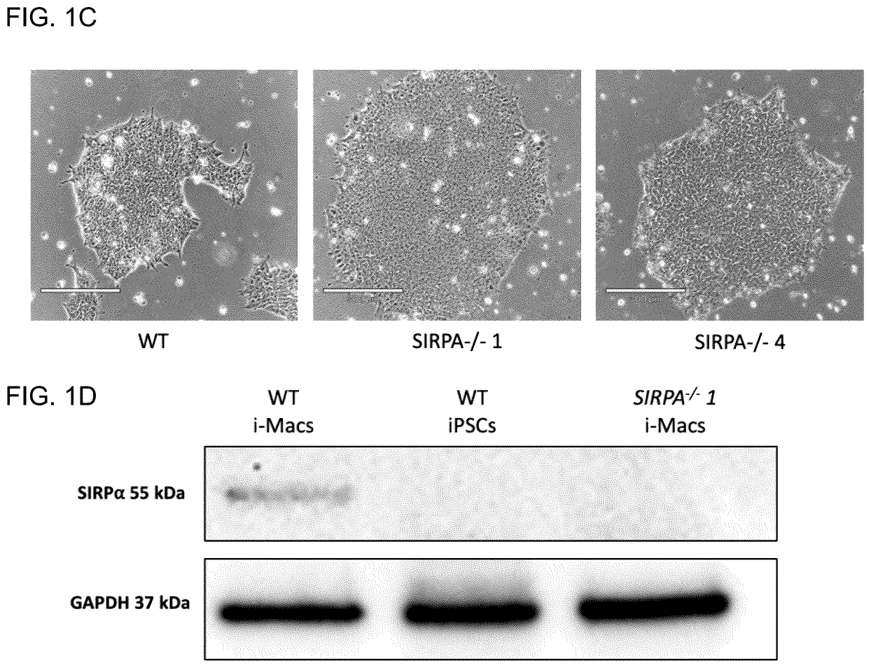

To develop the SIRPα-Knockout iPS cell line, exon 3 of the SIRPA gene was targeted with two flanking sgRNAs. The CD47 binding region of SIRPα lies on exon 3, making it an ideal target for a functional SIRPα protein knockout (FIG. 1A). After electroporation and clonal selection, four independent SIRPα-KO clones were genotyped by PCR and expanded for further use (FIG. 1B). The morphology of the SIRPα-KO iPSC clones 1 and 4 resembled the WT iPSCs by forming adherent colonies on Matrigel coated plates (FIG. 1B), suggesting no off-target effects occurred at the level of pluripotency. To validate the SIRPα knockout, WT and SIRPα-KO1 iPSCs were differentiated into iPSC-Macrophages (i-Macs) and whole cell lysates were collected for immunoblotting. Due to the lack of SIRPα expression on stem cells, WT iPSCs were used as a negative control. SIRPα protein was absent in the SIRPα-KO i-Macs compared to WT i-Macs, substantiating the successful knockout of SIRPα.

### Hematopoietic Differentiation of SIRPα-KO iPSCs

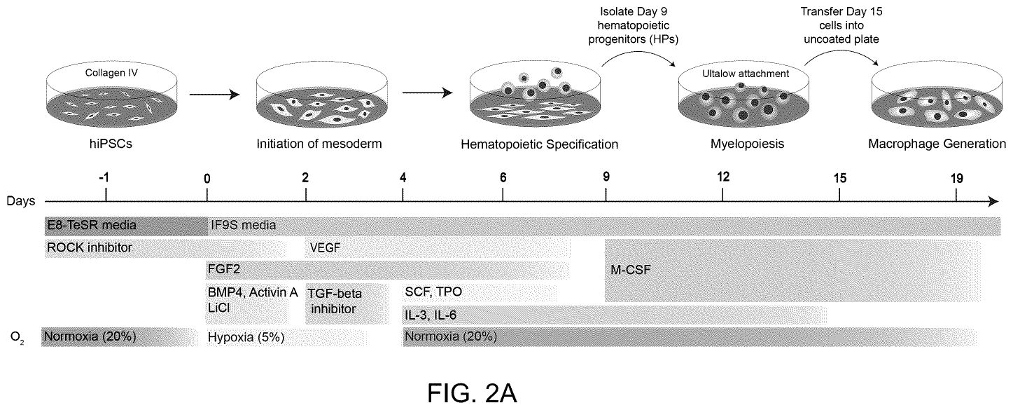

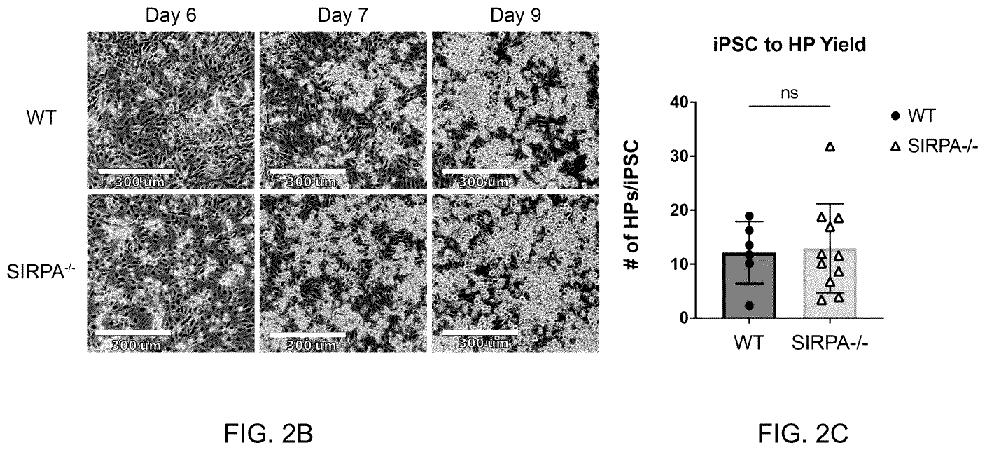

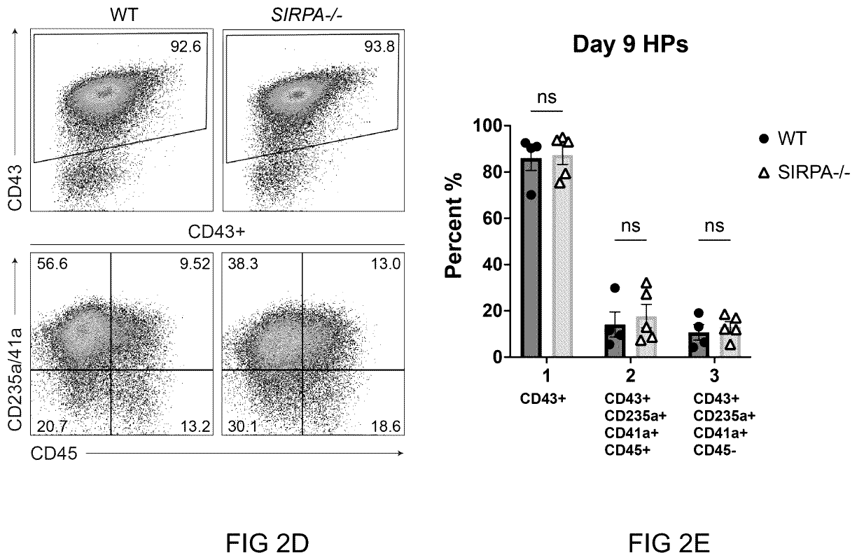

Previously, a serum-free, xeno-free, component-defined method was developed for in vitro hematopoietic differentiation that utilizes morphogen-driven formation of the hemogenic endothelium (Uenishi et al., 2014, Stem Cell Reports, 3: 1073-1084). These methods allow for the generation of multipotent hematopoietic progenitors (HPs) with lymphoid and myeloid potential by day 9. With adaptations to the protocol using similar myelopoiesis-driving cytokines disclosed in Cao et al., 2019, Stem Cell Reports, 12: 1282-1297, SIRPα-KO iPSC-macrophages (i-Macs) were generated in 19 days in serum-free conditions (FIG. 2A).

The generation of the hemogenic endothelium (HE) was observed in SIRPα-KO iPSC-HE with morphology resembling WT iPSC-HE (FIG. 2B). On day 5-6 of hematopoietic differentiation, the endothelial to hematopoietic cell transition was observed when round floating hematopoietic progenitors (HPs) arise from the adherent hemogenic endothelium, saturating the culture plate by day 9 (FIG. 2B). The number of HPs generated from a singular iPSC, or iPSC to HP yield, was discovered to be unaffected by the knockout of SIRPα (FIG. 2C). To further investigate the phenotype of SIRPα-KO iPSC-HPs, flow cytometry analysis of the floating cells was performed. CD43+ population of SIRPα-KO iPSC-HPs were around 93%, indicating the floating cells on day 9 were a nearly pure population of hematopoietic progenitors. When further probed, the CD43+ subpopulation of SIRPα-KO iPSC-HPs showed the same distribution of lineage markers CD235a/41a and CD45 as the WT iPSC-HPs (FIG. 2D). Together, these findings indicate that genetic knockout of SIRPα in iPSCs does not affect the specification to the hemogenic endothelium or generation of multipotent hematopoietic progenitors.

### Differentiation into SIRPα iPSC-Macrophages

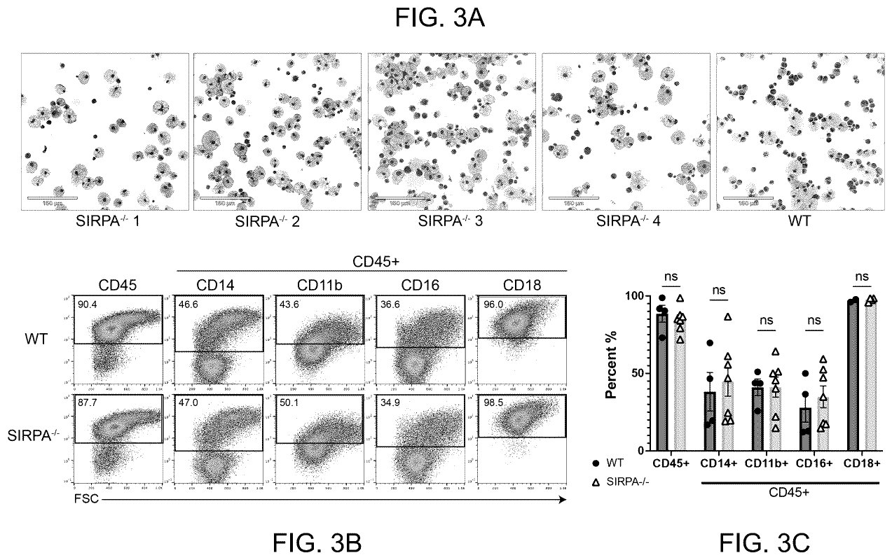

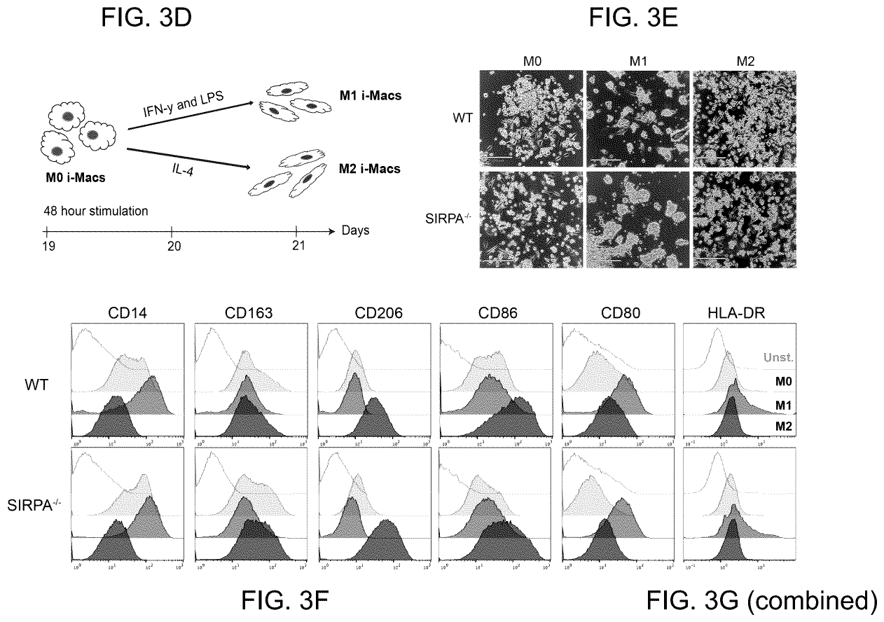

To generate macrophages from SIRPα-KO iPSC-HPs, the day 9 floating progenitor cells were collected and resuspended in IF9S media with M-CSF, IL-3, and IL-6. They were then plated on ultra-low attachment 6-well plates at a density of 1×10⁵ cells/well and placed in the normoxic incubator for 72 hours. On day 12, the media was replenished, and myeloid cells formed in clumps until day 15. Myelopoiesis is essential for the generation of macrophages, so analysis of the heterozygous SIRPα-KO iPSC-myeloid cells on day 15 is critical for understanding the myeloid potential of said cells. The morphology of the SIRPα-KO iPSC-myeloid cells showed the population is not purely iPSC-monocytes, but rather a mix of myeloid progenitors, granulocytes, and macrophages (FIG. 3A). These findings suggest that in vitro hematopoiesis does not give rise to a stage of pure peripheral blood mononuclear cells (PBMCs) as found in adult humans, but rather a heterozygous mix of myeloid progenitors that differentiate into macrophages at varying time points (FIG. 3A).

The day 15 SIRPα-KO iPSC-myeloid cells were investigated further through flow cytometry analysis. The CD45+ population of SIRPα-KO iPSC-myeloid cells was around 90%, indicating at this time-point, the cells were committed to the blood lineage (FIG. 3B and FIG. 3C). CD45+ subpopulation was analyzed for macrophage markers: CD14, CD11b, CD16, and pan-myeloid marker CD18. The CD14+CD11b+CD16+ population of SIRPA-KO iPSC-myeloid cells was around 40%, meaning on day 15 of differentiation, approximately 40% of the cells were committed to macrophages, also seen in the morphology of the larger, foamy cytoplasmic cells in the stained sample indicated by the arrowheads (FIG. 3A, FIG. 3B, and FIG. 3C). SIRPα-KO iPSC-myeloid cells resembled the same morphology and myeloid markers as WT iPSC-myeloid cells, suggesting that knocking out SIRPα had no effect on myelopoiesis.

### SIRPα-KO iMacs Polarize in Response to Stimuli

Macrophages have impressive diversity and plasticity, depending on the environment they reside. Based on cues that macrophages receive from their environment, they possess the ability to polarize into an array of phenotypes which differ in function and morphology. Classically activated macrophages, or M1 macrophages (M1-Macs), are stimulated by pathogens during infection and pro-inflammatory cytokines secreted from other immune cells. They possess pro-inflammatory and anti-tumorigenic capabilities, and highly express markers CD80, CD86 and antigen-presenting machinery HLA-DR. Alternatively activated macrophages, or M2 macrophages (M2-Macs), are stimulated by an immunosuppressive milieu and cytokines IL-4, IL-10 and TGF-beta. M2-Macs highly express markers CD163 and CD206 and are responsible for wound healing and regeneration of tissues, and therefore are anti-inflammatory. In the context of the TME, immunosuppressive signaling alters the TAM phenotype towards an M2, pro-tumorigenic state.

To test the plasticity and response to stimuli, day 19 SIRPα-KO i-Macs 19 cells were polarized into M1 and M2 phenotypes by the addition of IFN-γ+LPS and IL-4, respectively, for 48 hours (FIG. 3D). The morphology of the WT and SIRPα-KO i-Macs were visibly altered after the polarization period, notably the M1 polarized i-Macs were tightly clumped and adhered to the bottom of the plate, while the M2 polarized i-Macs were mostly floating and displayed only a few elongated and adhered cells (FIG. 3E). By utilizing flow cytometry analysis, the cell surface expression of M1 markers, CD86 and CD80, and M2 markers, CD163 and CD206 of the i-Macs were analyzed post-polarization. Surprisingly, M2-polarized WT and SIRPα-KO iMacs had the highest expression of the M1-marker CD86 and no upregulation of M2-marker CD163 (FIG. 3F). This finding suggests that hiPSC-Macrophages may possess a more pro-inflammatory phenotype even when stimulated with IL-4. This may be an advantage for the clinical use of SIRPα-KO i-Macs against solid tumors as they may be resistant to the immunosuppressive milieu containing IL-4 in the TME. However, the M2 SIRPα-KO i-Macs had an expected increased expression of CD206 and downregulation of CD14 and CD80 (FIG. 3F). M1-polarized SIRPα-KO i-Macs upregulated CD14 and CD80 expression, compared to both M0 and M2 phenotypes. Most interestingly, M1 polarization increases HLA-DR expression on the i-Macs, implicating that SIRPα-KO i-Macs possess antigen-presentation machinery and the ability to communicate with the adaptive immune system (FIG. 3F). This suggests with the TME, activated SIRPα-KO i-Macs would have the potential to present phagocytosed tumor antigen to nearby T cells. Overall, these data display that SIRPα-KO i-Macs upregulate and/or downregulate the same cell surface markers as WT i-Macs in response to M1 and M2 polarization, validating that knocking out SIRPα has no effect on macrophage plasticity or response to stimuli.

### SIRPα-KO i-Macs Have Superior Anti-Tumorigenic Properties Against CD47+ Cancer Cells

The most crucial qualities of SIRPα-KO i-Macs to assess is their ability to phagocytose cancer and overall anti-tumorigenic properties. CD47 is overexpressed by many solid tumor cancers including ovarian, pancreatic, breast, lung, and melanoma cancers, and subsequently blocks macrophage phagocytosis. It would be expected to see an increase of phagocytosis by SIRPα-KO i-Macs against CD47-expressing cancer cells. However, because CD47/SIRPα signaling only blocks an activating signal, simply knocking out SIRPα should not initiate non-specific phagocytosis. Thus, to direct the SIRPα-KO i-Macs to phagocytose cancer cells, therapeutic monoclonal antibodies (mAb) were used. The Fc-receptors on macrophages bind to mAbs that target specific cancer-antigens and stimulate antibody-dependent cellular phagocytosis (ADCP), consequently killing the tumor cells.

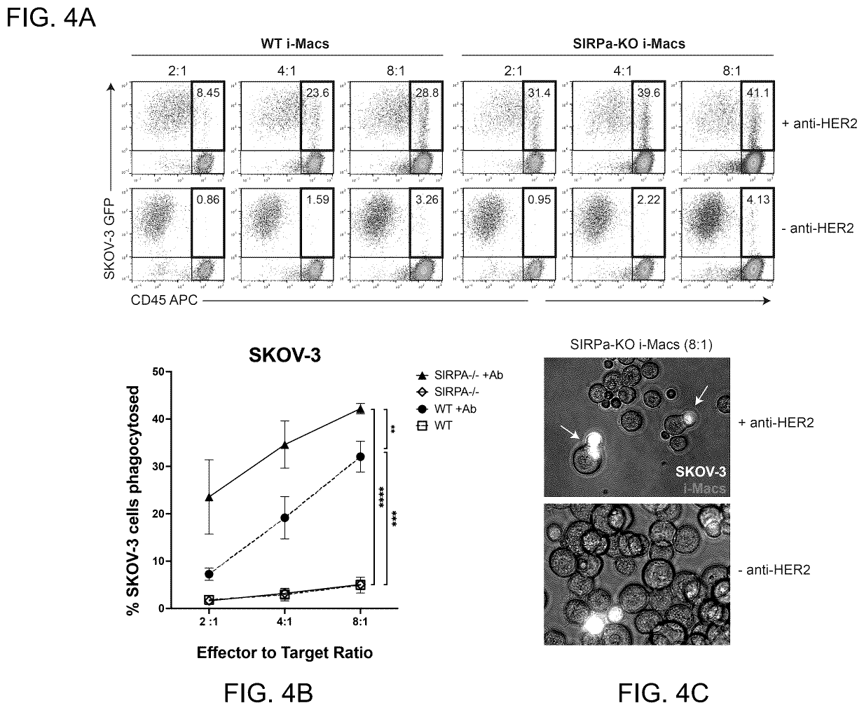

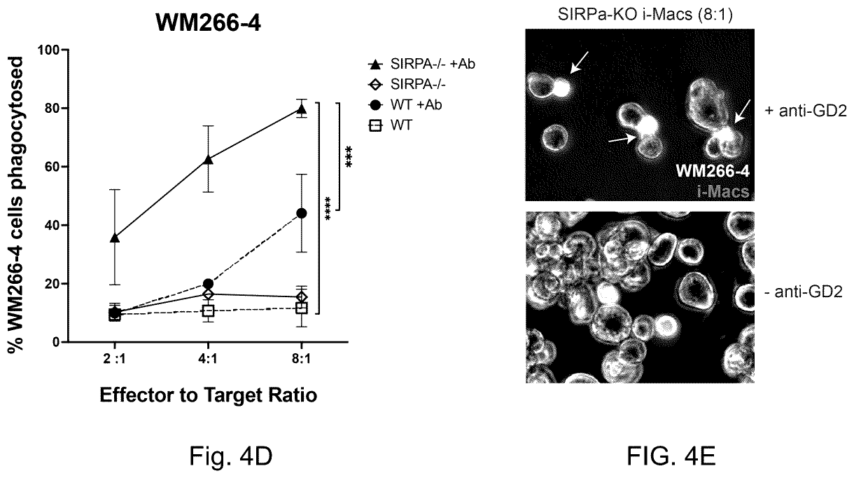

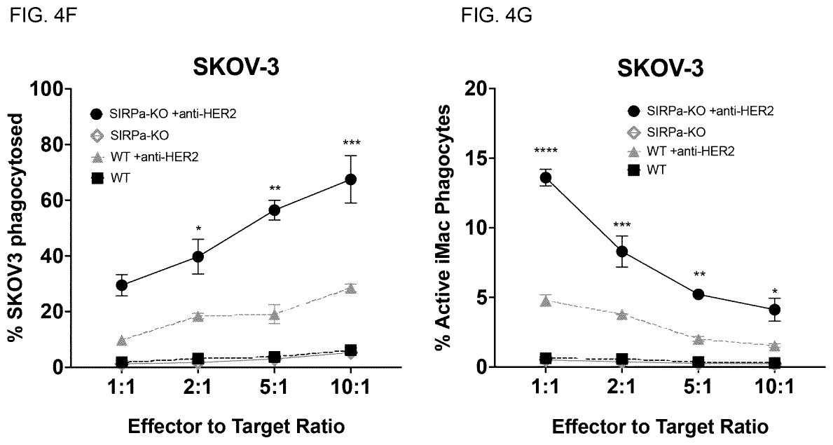

CD47 overexpression has previously been shown to predict poor prognosis in patients and promote cancer cell invasion in high-grade serous ovarian carcinoma and is expressed on ovarian cancer cell line SKOV-3 (Li, et al., American Journal of Translational Research, 9(6), 2901-2910). In an in vitro cancer challenge, SKOV-3 GFP+ ovarian cancer cells were cultured with WT or SIRPα-KO i-Macs in varying effector to target (E:T) ratios with or without anti-HER2 for 24 hours. Without the addition of anti-HER2, both the WT and SIRPα-KO i-Macs only phagocytosed a maximum of 3-4% of the cancer cells at 8:1 effector to target ratio (FIG. 4A and FIG. 4B). When anti-HER2 was added to the 24-hour co-culture, the SIRPα-KO i-Macs phagocytosed around 25%, 35% and 42% of the SKOV-3 cells at a 2:1, 4:1 and 8:1 effector to target ratio, respectively; significantly more than the WT i-Macs phagocytosis with anti-HER2 across all ratios (FIG. 4A and FIG. 4B). The SIRPα-KO i-Macs visibly attached to the SKOV-3 GFP+ cells in addition of the anti-HER2 mAb as seen by the white arrows in FIG. 4C. Even with merely 2:1 effector to target ratio, SIRPα-KO i-Macs phagocytosed nearly 3× the WT i-Macs with antibody.

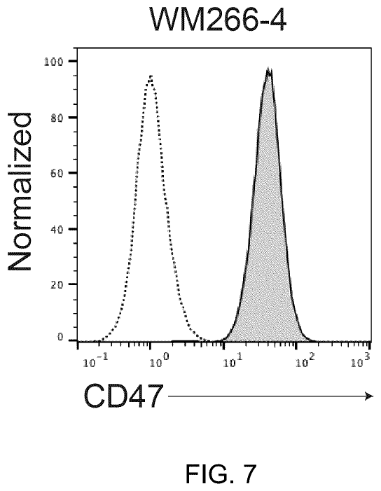

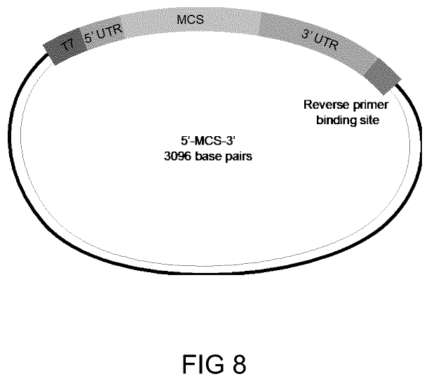

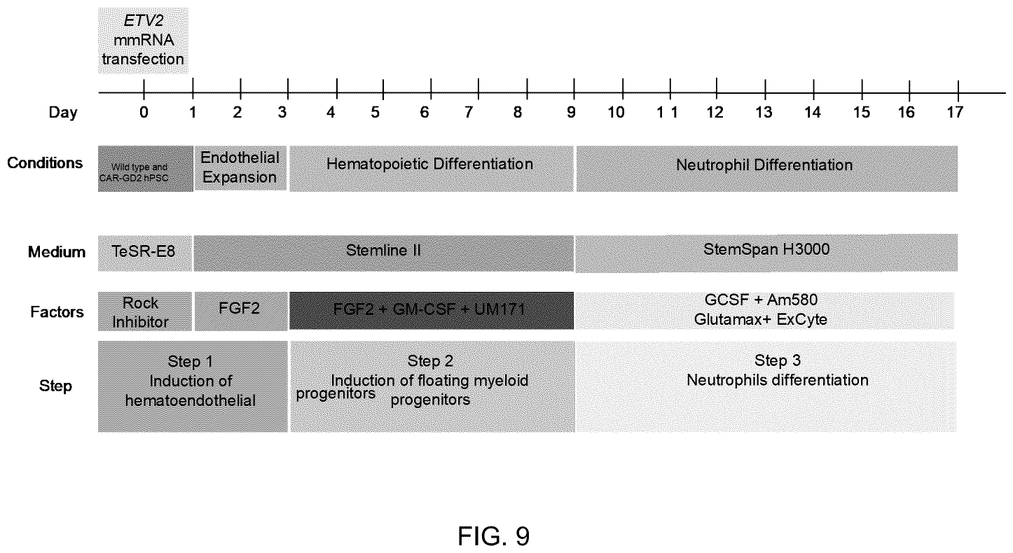

To test the phagocytic capability across multiple cancers, GD2-expressing WM266-4 melanoma cancer cells stained with CellTrace Violet were co-cultured with i-Macs with or without anti-GD2 Ch14.18 mAb. Since WM266-4 highly express CD47, similar results of enhanced phagocytosis by the SIRPα-KO i-Macs (FIG. 7) were expected. Flow cytometry was performed to quantify double-positive CellTrace Violet+CD14+ phagocytic macrophages. After only 6 hours, SIRPα-KO i-Macs+anti-GD2 phagocytosed approximately 80% of the WM266-4 cells at an 8:1 effector to target ratio, significantly more than WT i-Macs with or without anti-GD2 (FIG. 4D and FIG. 4E). These findings indicate that SIRPα-KO i-Macs have superior phagocytosis of multiple CD47-expressing cancers with the addition of cancer-specific therapeutic monoclonal antibodies.

To capture a smaller time frame for antibody-dependent phagocytosis (ADCP), HER2-expressing SKOV-3 GFP+ovarian cancer cells were cultured with wildtype (WT) or SIRPα-KO iMacs with or without anti-HER2 monoclonal antibody for a total of 6 hours. As seen in the 24-hour assay, without the addition of anti-HER2, both WT and SIRPα-KO iMacs phagocytosed little to none of the cancer cells at all effector-to-target (E:T) ratios, suggesting the safety of SIRPα-KO iMacs against healthy CD47+ cells (FIG. 4F). However, when anti-HER2 was added, the SIRPα-KO iMacs phagocytosed SKOV-3 cells at a significantly higher rate than WT (FIG. 4F). Additionally, at only 1:1 E:T ratio, SIRPα-KO iMacs have significantly higher percentage (%) of actively phagocytes than WT when anti-HER2 was added, with 13.6%±0.60 of SIRPα-KO iMacs phagocytosing compared to only 4.77%±0.41 of WT iMacs (FIG. 4G). These data suggested that knocking out SIRPα in iMacs triggered a larger proportion of macrophages to engage in phagocytosis when exposed to SKOV-3 cancer and anti-HER2 mAb, as opposed to WT iMacs, resulting in higher % of SKOV-3 cells to be depleted by antibody-dependent phagocytosis in as little as 6 hours.

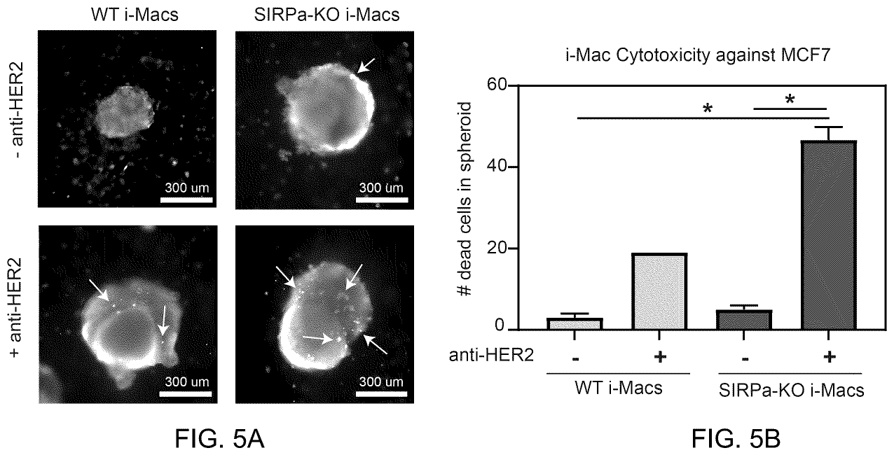

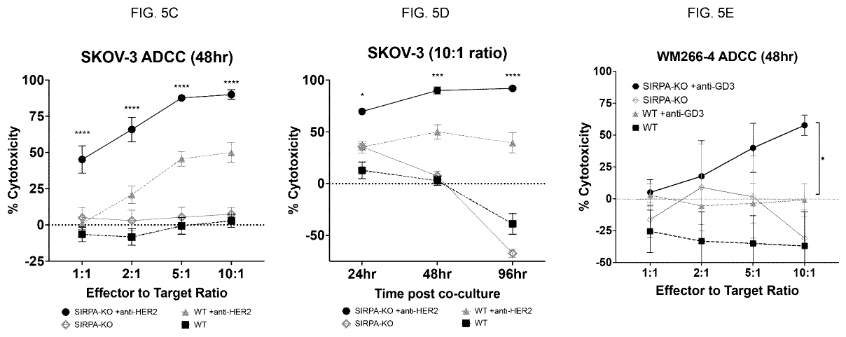

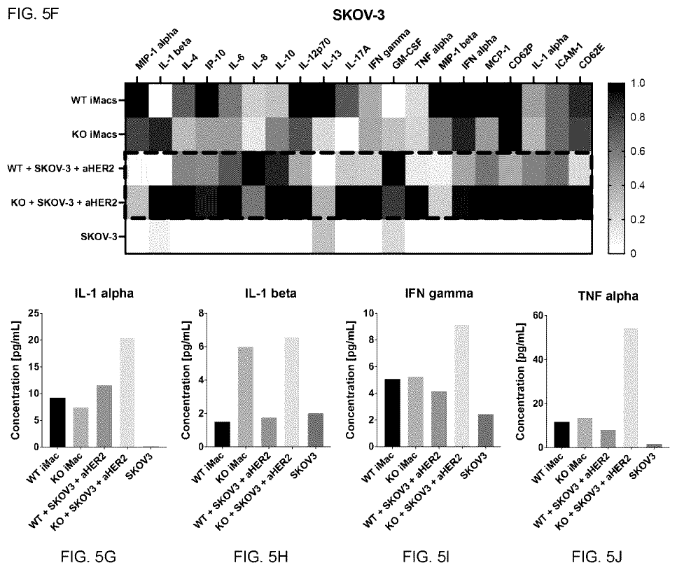

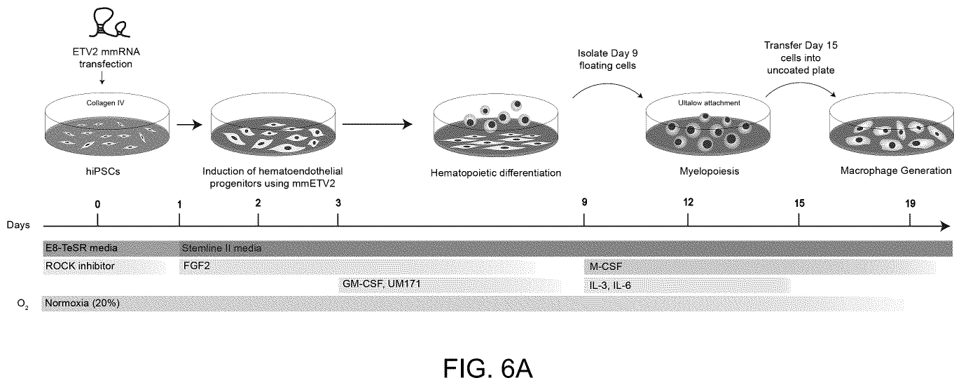

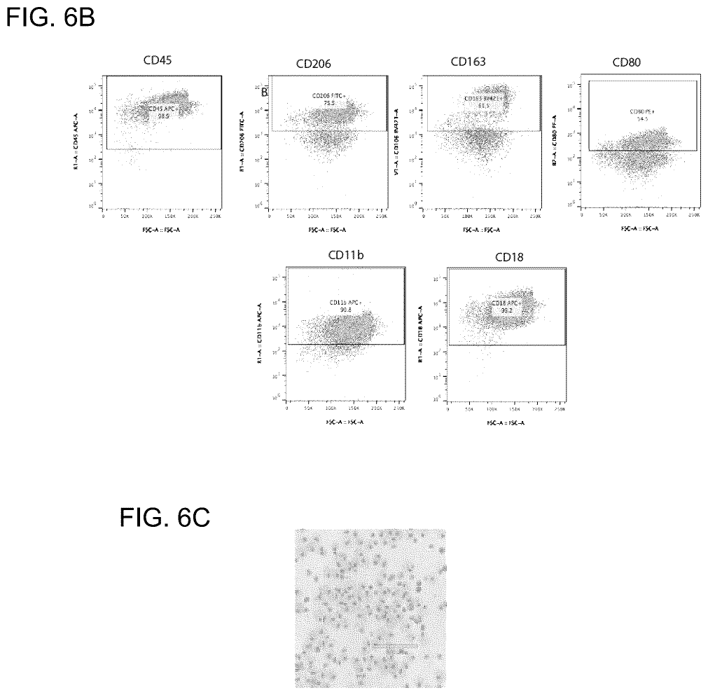

Macrophages also have the capability for cytotoxicity through antibody-dependent cellular cytotoxicity (ADCC), an appealing quality for cancer immunotherapy. When SIRPα-KO i-Macs+anti-HER2 mAb were co-cultured with HER2+CD47+MCF7 breast cancer spheroids for 4 days, there were significantly more dead cells, as shown by the white arrows, within the MCF7 cancer spheroid than wildtype (WT) counterparts (FIG. 5A and FIG. 5B). There was little to no cytotoxicity in the cancer cells without the anti-HER2 monoclonal antibody in either SIRPα-KO or WT i-Macs (FIG. 5A and FIG. 5B). These preliminary data suggested that CD47/SIRPα signaling blocked more than just an activating phagocytic signal, but also blocked any antibody-dependent anti-tumorigenic properties from macrophages. Together, these findings suggested that the novel SIRPα-KO i-Macs are an attractive candidate for clinical treatment of solid tumors and may be used as an inducible in combination with therapeutic monoclonal antibodies.

Current in vitro antibody-dependent phagocytosis assays only consider the GFP+ cells within CD45+ iMacs during flow cytometric analysis and disregard any cancer cells that were killed or destroyed by mechanisms other than phagocytosis. To assess in vitro ADCC, iMacs were co-cultured with a fixed number of luciferase-expressing SKOV-3 cells over varying E:T ratios and time points, bioluminescence using SpectraMax plate reader serving to quantify the viability of SKOV-3 cells. Percent (%) cytotoxicity was calculated by the following formula:

100×(baseline tumor growth−experimental value)/(baseline−maximum lysis control).

In these experiments, SIRPα-KO iMacs were significantly better at killing SKOV-3 cells under any condition with addition of anti-HER2 as compared to wildtype (WT) iMacs+anti-HER2 (FIG. 5C and FIG. 5D). After 96 hours post co-culture, SIRPα-KO iMacs killed 93%±1.47 of SKOV-3 cells in 96hrs compared to 27.3%±8.73 SIRPα-KO iMacs that killed nearly 100% of the ovarian cancer (FIG. 5D). This data also showed that within the confines of an in vitro assay, SIRPα-KO iMacs with anti-HER2 mAb were able to overcome inherent growth of the ovarian cancer over 96 hours that was witnessed when SIRPα-KO or WT iMacs were co-cultured with SKOV-3 without antibody, represented as negative % cytotoxicity in FIG. 5D.

To test SIRPα KO iMacs' cytotoxic capacity across multiple cancer types, GD2+GD3+WM266-4 melanoma cancer cells were co-cultured with iMacs with or without anti-GD3 mAb for 48 hours. With the addition of anti-GD3, SIRPα-KO iMacs displayed significantly higher cytotoxicity than WT iMacs at the 10:1 ratio, and showed steady increase of cytotoxicity across all ratios, unlike any other iMac group (FIG. 5E). WT iMacs instead displayed “negative” cytotoxicity, representing the growth of the WM266-4 melanoma cells from baseline. These data indicated that the tumor-promoting quality of WT iMacs that SIRPα-KO iMacs do not possess. To conclude the in vitro ADCC assays, SIRPα KO iMacs were challenged with 3D MCF-7 spheroids, a low-HER2-expressing breast cancer cell line. After 4 days of co-culture, SIRPα-KO iMacs with anti-HER2 killed a higher number of tumor cells within the spheroid than WT+anti-HER2, supporting that SIRPα-KO iMacs have increased capacity to infiltrate and kill cancerous cells within a 3D TME structure, even against tumors with low antigen expression (FIG. 5A-FIG. 5B). This spheroid model was particularly useful because it is both quantitative and spatially qualitative. These results showed that in vitro, SIRPα-KO iMacs had superior antibody-dependent antitumor activities, including phagocytosis and cytotoxicity against SKOV-3, WM266-4, and MCF-7 solid tumor cancer cell lines, and enhanced the potency of FDA-approved monoclonal antibody therapies.

Macrophages within the tumor microenvironment have been implicated in promoting growth, metastases, and survival of solid tumors through a multitude of mechanisms including direct cell-to-cell signaling, and cytokine signaling (Chen et al., 2019, Journal of Biomedical Science 26: 78; Chanmee et al., 2014, Cancers 6: 1670-1690; and Zhu et al., 2021, Journal of Cancer 12: 54-64). Many anti-inflammatory cytokines and factors expressed by macrophages such as TGF-beta, IL-10, Arg1, IDO, and HIF-1alpha have been associated with solid tumor progression. Due to the myriad of anti-inflammatory cytokine expression, pro-inflammatory cytokines that promote activation of immune cells to an anti-tumorigenic state are suppressed or over-powered. SIRPα- and CD47-signaling between tumor associated macrophages within the tumor microenvironment has not been thoroughly assessed in the context of cytokine signaling pathways. To evaluate if the SIRPα-KO iMacs possessed the same cytokine signaling in response to tumor as WT iMacs, iMacs and SKOV-3 ovarian cancer cells were co-cultured together for 48 hours and 20 different cytokines were evaluated through a multiplex assay (FIG. 5F). Surprisingly, SIRPα-KO iMacs were found to upregulate many pro-inflammatory cytokines in the presence of anti-HER2 mAb and SKOV-3 tumor as compared to WT iMacs. Specifically, IL-1alpha, IL-1beta, IFN-gamma, and TNF-alpha were all upregulated in the SIRPα-KO iMac+anti-HER2 treatment group (FIG. 5G through 5J, respectively). These data suggested that SIRPα may play a larger role in the macrophage-tumor axis than just direct cell to cell blocking of phagocytosis. Another possibility is that without the presence of SIRPα, macrophages are more readily engaging with antibody-dependent tumor killing or phagocytosis and have subsequent changes in the cytokines being released within the milieu. These results indicated an increased expression of pro-inflammatory cytokines in the SIRPα-KO iMacs+anti-HER2 group as compared to WT+anti-HER2, further supporting the enhancement of antibody-dependent functions within SIRPα-KO iMacs.

### Example 2: Generation of SIRPA Knockout Neutrophils and Characterization Thereof

### Materials and Method

### Cell Culture

Wild type bone marrow-derived IISH2i-BM9 hiPSCs (Hu et al., 2011, eBlood 117: e109-e119) was obtained from WiCell (Madison, WI). Human induced pluripotent stem cells (hiPSCs) with knockout SIRPA gene were generated using CRISPR/Cas9 technology. Wild type and SIRPA^{-/-} hiPSCs were cultured on Matrigel-coated tissue culture plates in E8 medium (STEMCELL Technologies).

### mmRNA Synthesis and Transfection

Human ETV2 transcript variant 1 (NM_014209.3) was cloned into a 5′-MCS-1β construct as described previously (Suknuntha et al., 2018, Stem Cell Rev. 14: 525-534). To generate IVT templates with a 180-A tract, a reverse primer containing 180 T base pairs and an ATCGGTGCGGGCCTCTTCGCTA (SEQ ID NO: 3) forward primer including T7 promoter were used in a PCR reaction. All PCR reactions were carried out using Phusion (Thermo Fisher Scientific). The mmRNA was synthesized using the MEGAscript T7 Kit (Ambion, Austin, TX), using a custom ribonucleoside cocktail comprised of 3′-0-Me-m7G(5′)ppp(5′)G ARCA cap analog, pseudouridine triphosphate (TriLink BioTechnologies, San Diego, CA), ATP, guanosine triphosphate, and cytidine triphosphate. The synthesis reactions were set up according to the manufacturer's instructions. Reactions were incubated for 2 h at 37° C. and treated with DNAse. RNA was purified using a PureLink RNA Mini Kit (Thermo Fisher Scientific) and adjusted with RNase-free water to 100 ng/μL working concentration before being stored at −80° C. Undifferentiated hiPSCs were transfected with using TransIT-mRNA reagent in E8 medium containing ROCK inhibitor (Suknuntha et al., 2018, Stem Cell Rev. 14: 525-534). In brief, for transfection, single-cell suspension was prepared using HyQtase (Thermo Fisher Scientific). Per one well of transfection, a total of 2×10⁵ cells in 1 mL complete E8 medium with 10 μM ROCK inhibitor (STEMCELL Technologies) were plated into a collagen IV-coated 6-well plate; 30-60 min later, a mixture of 200 ng ETV2: TranslT-mRNA (Minis Bio, Madison, WI) was added to each well according to the manufacturer's instructions.

### Feeder-, Xeno-, and Serum-Free Generation of Neutrophils from hiPSCs

The day after transfection (day 1), the medium was changed with 1 mL of Stemline II (Sigma) supplemented with 20 ng/mL of human FGF2 (PeproTech). On day 2, 1 mL of the same medium was added. On day 3, the medium was changed and 1 mL of Stemline II supplemented with FGF2 (20 ng/mL), GM-CSF (25 ng/mL) (PeproTech), and UM171 (50 nM; Xcess Biosciences) were added. This medium was added daily up to days 8. On day 9, floating cells were gently harvested and used for terminal neutrophil differentiation. Following the first collection of floating cells, 2 mL of Stemline II supplemented with FGF2, GM-CSF, and UM171 was added to the remaining adherent cells. To induce neutrophil terminal differentiation, floating cells were cultured in StemSpanH300 medium (STEMCELL Technologies), supplemented with GlutaMAX 100X (Thermo Fisher Scientific), ExCyte 0.2% (Merck Millipore), human G-CSF (150 ng/mL; Amgen), Am580 retinoic acid agonist 2.5 μM (Sigma-Aldrich), and gentamycin (1,000×) (Life Technologies) at 5×10⁵ cells/mL density. After 4 days, 2 mL of the same medium with all components and cytokines was added on the top of existing culture. Mature neutrophils were gently harvested from the supernatant after 8 days of culture, leaving the adherent macrophages, and filtered through a 70-μM mesh (Falcon, Life Sciences) before analysis.

### Flow Cytometry

To analyze cell surface markers, 5×10⁵ cells were stained in fluorescence-activated cell sorting buffer with the appropriate antibodies (Table 1). Ghost Dye (Tonbo Biosciences, San Diego, CA) was used to analyze the live cell population. Cells were analyzed using a MACS Quant Analyzer 10 (Miltenyi Biotec, San Diego, CA) or Thermo Fisher Scientific FlowJo software (Tree Star, Ashland, OR).

TABLE 1

| ANTIBODIES | SOURCE |
|---|---|
| Mouse anti-human CD45 APC | BD Pharmingen ™ |
| Mouse anti-human CD11b FITC | BD Pharmingen ™ |
| Mouse anti-human CD16 PE | BD Pharmingen ™ |

### Wright-Giemsa Staining

To assess the morphology of cells within colonies, cells were fixed on glass slides using a Cytospin centrifuge (Cytospin 2; Thermo Shandon), stained with Wright-Giemsa solution (Sigma-Aldrich), and then observed under a light microscope (Olympus, Tokyo).

### In Vitro Cytotoxicity Assay

WM266-4 LUC2 GFP, CHLA-20_AAVS1-AkaLuc-EGFP, SK-BR3 LUC2 GFP and SKOV3 LUC2 GFP cells were maintained in tumor specialized media (Sigma-Aldrich) containing 80% MCDB-153, 20% Leibovitz's L-15, 1.68mM CaCl2 and 2% FBS. To access the cytotoxicity, hiPSC derived wild type and SIRPA^{-/-} neutrophils or SIRPα-KO neutrophils or macrophages were incubated with target tumor cells (2,000 cells/well) for 4 hours at 37° C., at effector: target (E:T) ratios of 1:1, 2:1, 5:1, and 10:1, in a final volume of 200 μL, in a 96 well plate. Target cells were used for the maximal lysis with Pierce™ IP Lysis Buffer (ThermoFisher). VivoGlo™ Luciferin substrate (100 ug/well, Promega) was added, and luminescence was measured immediately after 5 mins incubation. Specific cell lysis was measured by % of cell lysis=100×{(Spontaneous death relative luminescence unit (RLU)−Test RLU)/(Spontaneous death RLU−Maximal killing RLU)}.

### Candida auris Planktonic Yeast Kill Assay

Wild type and SIRPA^{-/-} neutrophils were plated at a 1:1 MOI for six hours. hiPSC neutrophils were then lysed with ddH2O plus 100 ug/ml DNase 1 solution for 1 hour. PrestoBlue (metabolic assay) reagent was then added and incubated for 2 hours at 37c/5% CO2 before reading fluorescence at 560/590 nm. The percentage of viable yeast was quantified relative to yeast with no neutrophils. Neutrophil-only controls were subtracted from that value. Bar graph show±SEM from 3 independent experiments showing percent yeast survival. Differences compared to wild type were not significant (p=0.1755) as determined by unpaired t test.

### Phagocytosis

Phagocytosis was assessed using pHrodo Green E. coli BioParticles Conjugate (Invitrogen) according to a modified manufacturer's protocol. pHrodo Green E. coli beads were resuspended in 2 mL of PBS and sonicated with an ultrasonicator 3 times (20% amplitude, 20 s on/10 s off). Beads per assay (100 μL) were opsonized by mixing with opsonizing reagent at a 1:1 ratio and incubated at 37° C. for 1 h. Beads were washed 3 times mHBSS buffer by centrifugation at 4° C., 1,500 RCF for 15 min then final resuspension in mHBSS buffer. Beads were used immediately or stored at 4° C. for several days. Wild type and SIRPA^{-/-} neutrophils (5×10⁵) were resuspended in 100 μL of opsonized bead solution and incubated at 37° C. or on ice for 1 h. Phagocytosis was stopped by placing all samples on ice. Analysis was carried out with Thermo Fisher Scientific Attune cytometer for fluorescent particles (509/533). Cells were gated based on granulocyte population, single cells, and live cells using propidium iodine.

### Measurement of Reactive Oxygen Species Production in Neutrophils

Floating wild type and SIRPA^{-/-} neutrophils (10⁵) were plated in each well of a black 96-well plate on 10 μg/mL fibrinogen with 100 μL of mHBSS buffer in the presence of 10 ng/mL dihydrorhodamine 123. Cells were incubated for 30 min at 37° C./5% CO2. PMA was added to a final concentration of 50 ng/mL or vehicle control DMSO was added to samples. Optimal reactive oxygen species production was determined by time course. Fluorescent measurements were taken of samples in triplicate or replicates of four on the Victor3 V plate reader (Ex/Em 500/536).

### Chemotaxis Assay

Chemotaxis was assessed using a microfluidic device. In brief, polydimethylsiloxane devices were plasma treated and adhered to glass coverslips. Devices were coated with 10 μg/mL fibrinogen (Sigma) in PBS for 30 min at 37° C., 5% CO2. The devices were blocked with 2% BSA-PBS for 30 min at 37° C., 5% CO2, to block non-specific binding, and then washed twice with mHBSS. Wild type and SIRPA^{-/-} neutrophils were stained with calcein AM (Molecular Probes) in PBS for 10 min at room temperature followed by resuspension in modified Hank's balanced salt solution (mHBSS). Wild type and SIRPA^{-/-} neutrophils were seeded at 5×10⁶/mL to allow adherence for 30 min before addition of chemoattractant. Either 1 μM fMLP (Sigma) or 11.25 μM IL-8 (R&D Systems) was loaded onto the devices. Cells were imaged for 45-90 min every 30 s on a Nikon Eclipse TE300 inverted fluorescent microscope with a 10×μ objective and an automated stage using MetaMorph software (Molecular Devices). Automated cell tracking analysis was done using JEX software to calculate chemotactic index and velocity.

### Human Inflammation 20-Plex ProcartaPlex Panel

SIRPα-KO and wildtype (WT) iMacs were either cultured alone or co-cultured with SKOV-3 ovarian cancer and anti-HER2 monoclonal antibody for 48 hrs. The supernatants were collected for Human Inflammation 20-Plex ProcartaPlex Panel (ThermoFisher). Supernatants were tested in technical duplicates and raw values were produced on MAGPIX xMAP instrument plotted against a standardized curve.

### Results

### Morphology

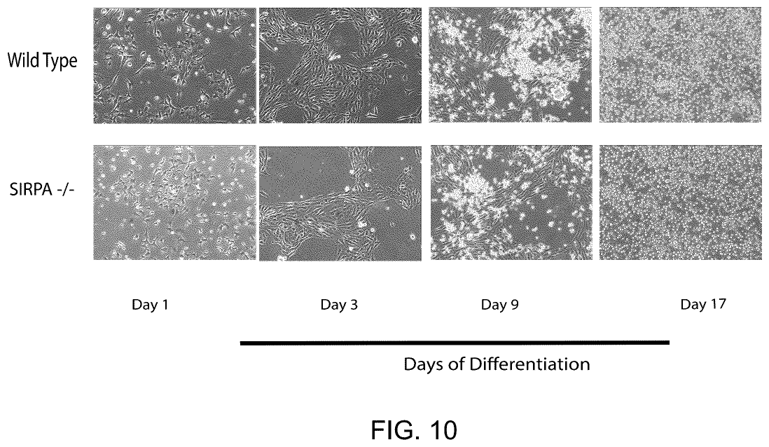

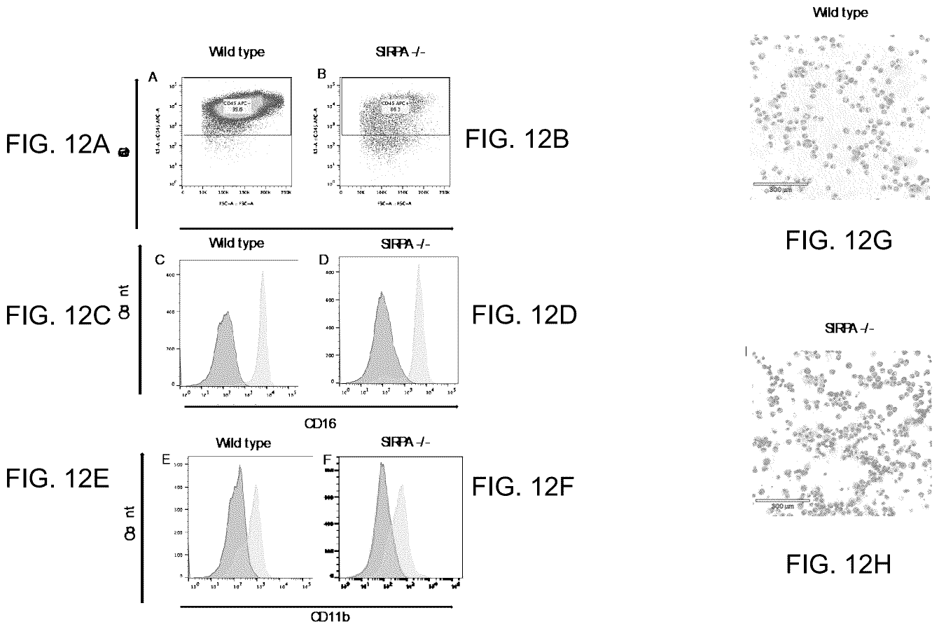

Transfection of ETV2 mmRNA into the single cell of wild type and SIRPA^{-/-} hiPSCs formed a typical endothelial morphology within 24 hours (FIG. 10). During the process of differentiation, cell changes it morphology and form floating myeloid progenitors (FIG. 10). After terminal differentiation, neutrophil was identified by Wright-Giemsa staining (FIG. 12).

### Cell Surface Markers

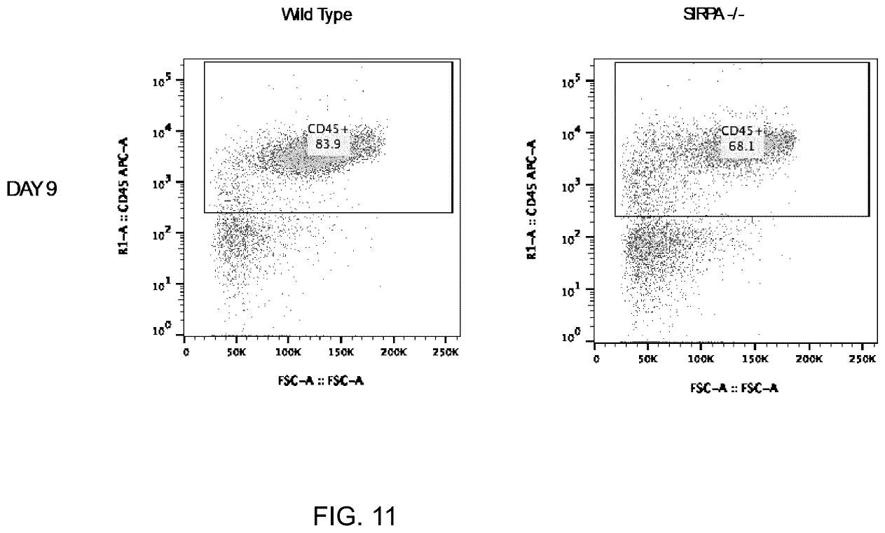

The myeloid progenitors expressed CD45 surface markers on day 9 (FIG. 11) in wild type and SIRPA^{-/-} cells. CD11b and CD16 expression in terminal differentiated neutrophils proved ETV2 mmRNA induced granulocytic differentiation program (FIG. 12).

Functional Evaluations

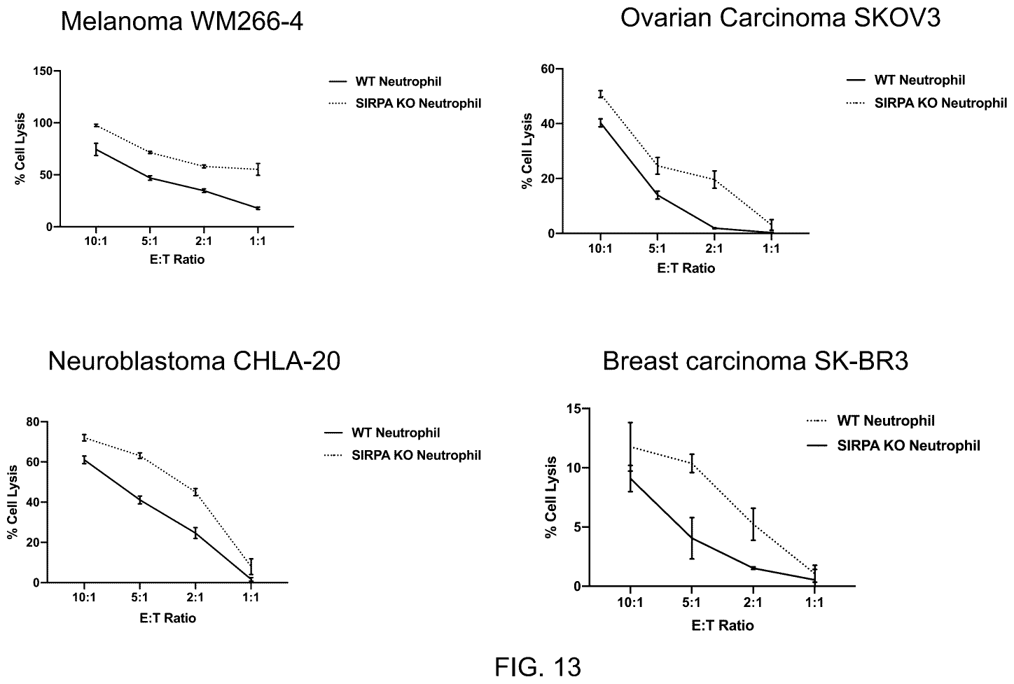

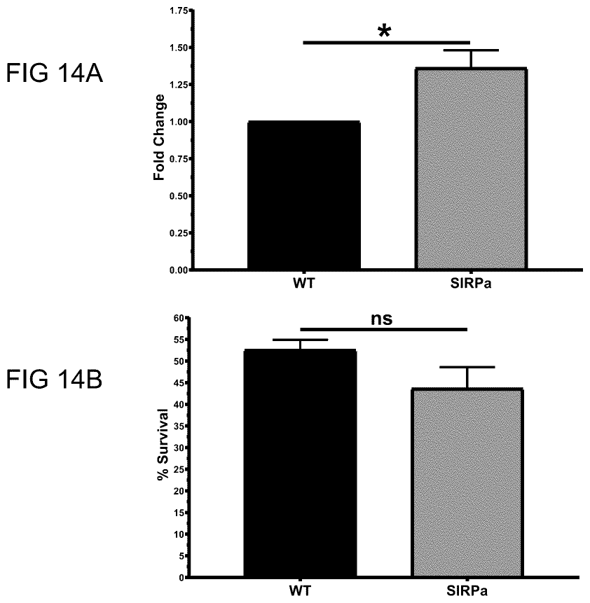

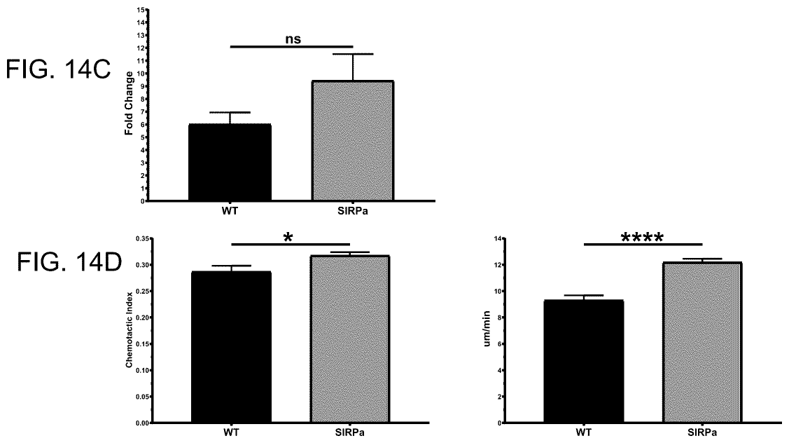

Functional characterization of neutrophils was performed using cytotoxicity assay (FIG. 13), phagocytosis and migration assay (FIG. 14). As compared to wild type neutrophils, SIRPA^{-/-} neutrophils demonstrated superior motility and phagocytic capacity of bacterial particles and tumor cells.

### Quantification

ETV2 mmRNA induction produced 1.7×10⁷ neutrophils from 10⁶ wild type hiPSCs and 3×10⁷ neutrophils from 10⁶ SIRPA^{-/-} hiPSCs within 3 weeks.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description can be made without departing from the spirit or scope of the present invention, as defined in the following claims.

1. A method of producing modified macrophages from pluripotent stem cells, the method comprising:
(a) culturing human pluripotent stem cells having inhibited expression of signal regulatory protein alpha (SIRPα) in normoxic conditions for about 24 hours in serum-free culture medium comprising of L-ascorbic acid-2-phosphate magnesium, sodium selenium, transferrin, insulin, NaHCO₃, fibroblast growth factor 2 (FGF2), transforming growth factor beta 1 (TGFβ1), and a Rho kinase (ROCK) inhibitor;
(b) further culturing the human pluripotent stem cells of (a) in hypoxia conditions for about 48 hours in serum-free culture medium comprising bone morphogenetic protein 4 (BMP4), FGF2, Activin A, an inhibitor of glycogen synthase 3 (GSK3), and a ROCK inhibitor to induce mesoderm formation;
(c) further culturing the cultured cells of (b) in hypoxic conditions for about 48 hours in serum-free culture medium comprising FGF2, a vascular endothelium growth factor (VEGF), and an inhibitor of TGFβ-mediated signaling to induce hemogenic endothelium formation;
(d) further culturing the cultured cells of (c) in normoxic conditions for about 6 days in serum-free culture medium comprising FGF2, a VEGF, stem cell factor (SCF), thrombopoietin (TPO), interleukin-6 (IL-6), and interleukin-3 (IL-3), wherein the hemogenic endothelium differentiate into HPCs;
(e) culturing the HPCs of (d) in normoxic conditions for about 6 days in serum-free culture medium comprising macrophage colony-stimulating factor (M-CSF), IL-3, and IL-6 to obtain myeloid progenitors and monocytic cells; and
(f) further culturing the cultured cells of (e) in normoxic conditions for about 4 days in serum-free culture medium comprising M-CSF, whereby the cultured myeloid progenitors and monocytes differentiate into a cell population comprising modified macrophages.

2. The method of claim 1, wherein the inhibitor of TGFβ-mediated signaling is SB431542.

3. The method of claim 1, wherein the inhibitor of GSK3 is lithium chloride (LiCl).

4. The method of claim 1, wherein the ROCK inhibitor is Y-27632.

5. The method of claim 1, wherein the expression of SIRPα is inhibited in the human pluripotent stem cells by gene mutation, RNA-mediated inhibition, RNA editing, DNA gene editing or base editing.

6. The method of claim 5, wherein the gene editing method comprises the use of a nuclease selected from a meganuclease, zinc-finger nucleases (ZFNs), transcription activator-like effector nucleases (TALENs), and Cas enzyme.

7. The method of claim 5, wherein gene editing results in knocking out SIRPα expression.

8. The method of claim 6, wherein the nuclease is a Cas9 enzyme.

9. The method of claim 1, wherein the pluripotent stem cells are induced pluripotent stem cells.

10. A population of modified macrophages produced by the method of claim 1.

11. A method of producing modified macrophages from pluripotent stem cells, the method comprising:
(a) transiently introducing exogenous ETV2 in human pluripotent stem cells having inhibited expression of SIRPα and culturing the ETV2-induced pluripotent stem cells in serum-free culture medium comprising FGF-2 to produce a population of ETV2-induced hematoendothelial progenitor cells (ETV2-induced HEPs);
(b) culturing the ETV2-induced HEPs in serum-free and xeno-free culture medium comprising granulocyte-macrophage colony-stimulating factor (GM-CSF) and FGF2 for a sufficient time to produce non-adherent myeloid progenitors;
(c) culturing the non-adherent myeloid progenitors in serum-free and xeno-free culture medium comprising M-CSF, IL-6, and IL-3; and
(d) further culturing the cultured cells of (c) in serum-free and xeno-free culture medium comprising M-CSF for a sufficient time to differentiate the non-adherent myeloid progenitors into modified macrophages.

12. The method of claim 11, wherein the serum-free and xeno-free culture medium in (b) further comprises UM171.

13. The method of claim 11, wherein the expression of SIRPα is inhibited in the human pluripotent stem cells by gene mutation, RNA-mediated inhibition, RNA editing, DNA gene editing or base editing.

14. The method of claim 13, wherein gene editing results in knocking out SIRPα expression.

15. The method of claim 13, wherein the gene editing method comprises the use of a nuclease selected from a meganuclease, ZFNs, TALENs, and Cas enzyme.

16. The method of claim 15, wherein the nuclease is a Cas9 enzyme.

17. The method of claim 11, wherein the method comprises one or more of the following:
step (a) comprises culturing the ETV2-induced pluripotent stem cells for about 1 to 2 days;
step (b) comprises culturing the ETV2-induced HEPs for about 6 to 7 days; and
steps (c) and (d) comprise culturing the myeloid progenitor cells for about 9 to 10 days.

18. The method of claim 11, wherein the pluripotent stem cells are induced pluripotent stem cells.

19. A population of modified macrophages produced by the method of claim 11.

20. A method of producing modified neutrophils from pluripotent stem cells, the method comprising:
(a) transiently introducing exogenous ETV2 in human pluripotent stem cells having inhibited expression of SIRPα and culturing the ETV2-induced pluripotent stem cells in serum-free culture medium comprising FGF-2 to produce a population of ETV2-induced hematoendothelial progenitor cells (ETV2-induced HEPs);
(b) culturing the ETV2-induced HEPs in serum-free and xeno-free culture medium comprising GM-CSF and FGF2 for a sufficient time to produce non-adherent myeloid progenitors; and
(c) culturing the non-adherent myeloid progenitors in serum-free and xeno-free culture medium comprising granulocyte-colony stimulating factor (G-CSF) and retinoic acid receptor agonist for a time sufficient to differentiate the myeloid progenitors into neutrophils.

21. The method of claim 20, wherein the serum-free and xeno-free culture medium in (b) further comprises UM171.

22. The method of claim 20, wherein the expression of SIRPα is inhibited in the human pluripotent stem cells by gene mutation, RNA-mediated inhibition, RNA editing, DNA gene editing or base editing.

23. The method of claim 20, wherein gene editing results in knocking out SIRPα expression.

24. The method of claim 22, wherein the gene editing method comprises the use of a nuclease selected from a meganuclease, ZFNs, TALENs, and Cas enzyme.

25. The method of claim 22, wherein the nuclease is a Cas9 enzyme.

26. The method of claim 20, wherein the retinoic acid receptor agonist is AM580.

27. The method of claim 20, wherein the method comprises one or more of the following:
step (a) comprises culturing the ETV2-induced pluripotent stem cells for about 1 to 2 days;
step (b) comprises culturing the ETV2-induced HPCs for about 6 to 7 days; and
steps (c) and (d) comprising culturing the myeloid progenitor cells for about 8 to 9 days.

28. The method of claim 20, wherein the pluripotent stem cells are induced pluripotent stem cells.

29. A population of modified neutrophils produced by the method of claim 20.

30. A method of treating cancer comprising administering the modified macrophages of claim 10 or claim 18.

31. The method of claim 30 further comprising administering a tumor-specific antibody.

32. A method of treating cancer comprising administering the modified neutrophils of claim 29.

33. A method of treating bacterial infection comprising administering the modified neutrophils of claim 29.

34. The method of claim 33, wherein the bacterial infection is a systematic infection.

35. A method of treating cancer comprising administering the modified macrophages of claim 18.

36. The method of claim 35 further comprising administering a tumor-specific antibody.
